(19) United States
(12) Patent Application Publication
(10) Pub. No.: US 2011/0318415 A1
(43) Pub. Date: Dec. 29, 2011

(54) Hollow Gold Nanospheres (HAuNSs) and HAuNSs-Loaded Microspheres Useful in Drug Delivery

(76) Inventors: Chun Li, Missouri City, TX (US); Jian You, Houston, TX (US)

(21) Appl. No.: 13/255,476

(22) PCT Filed: Mar. 9, 2010
(86) PCT No.: PCT/US10/26605

§ 371 Date: Sep. 8, 2011

Related U.S. Application Data
(60) Provisional application No. 61/158,570, filed on Mar. 9, 2009.
(60) Provisional application No. 61/233,566, filed on Aug. 13, 2009.

(51) Int. Cl. A61K 9/14; B82Y 5/00

(52) U.S. Cl. 424/486; 424/489; 424/400; 977/700; 977/810; 977/906

(57) ABSTRACT

A near-infrared mediated drug delivery system comprising a plurality of microspheres made of polymeric material, each sphere containing a plurality of hollow gold nanospheres together with drug product, wherein upon NIR irradiation, the drug product is released from the microsphere.

### CROSS REFERENCE TO RELATED APPLICATIONS

This patent application claims priority to U.S. Patent Application Ser. Nos. 61/158,570 and 61/233,566 which are incorporated by reference herein in its entirety.

### STATEMENT REGARDING FEDERALLY SPONSORED RESEARCH OR DEVELOPMENT

This invention was made with government support under R01 CA119387, Near-Infrared Fluoresence Nanoparticles for Targeted Optical Imaging, awarded by National Cancer Institute (NCI). The government has certain rights in the invention.

### THE NAMES OF PARTIES TO A JOINT RESEARCH AGREEMENT

None.

### INCORPORATION-BY-REFERENCE OF MATERIAL SUBMITTED ON COMPACT DISC

None.

### BACKGROUND OF THE INVENTION

Nanocarriers currently on the market as drug delivery platforms for therapeutic agents are organic nanoparticles consisting of lipids and/or synthetic polymers. These nanocarriers include liposomes and lipid nanospheres. Davis, S. S., Coming of Age of Lipid-based Drug Delivery Systems, 56 (9), 1241-2, Adv Drug Deliv Rev (2004). Also, certain polymeric drug delivery systems are in clinical trials. Nishiyama, N. et al., Current State, Achievements, and Future Prospects of Polymeric Micelles as Nanocarriers for Drug and Gene Delivery, 112 (3), 630-48, Pharmacol Ther (2006); Li, C. et al., Polymer-Drug Conjugates: Recent Development In Clinical Oncology, 60 (8), 886-98, Adv. Drug Deliv. Rev. (2008). While progress has been made in the development of drug delivery systems using organic nanoparticles, much less progress has been made in the development of inorganic nanoparticle-based drug delivery systems.

Despite advances in controlled drug delivery, reliable methods for activating, high-resolution control of drug release are needed. Inorganic nanoparticles have several advantages to offer as drug carriers. Inorganic nanoparticles can be prepared to a definite size and exhibit multiple functions useful in medicine—for example, serving as exothermic reactors and contrast agents. On the other hand, organic nanoparticles such as liposomes and polymer nanospheres serve only as drug reservoirs.

Further, prior art inorganic nanoparticles useful in drug delivery require a cytotoxic surfactant for stability. For example, other additives often needed such as to crystallize rod-shaped gold particles from aqueous solutions are cytotoxic. Furthermore, tumor uptake of the drug is often ineffective because drug payload with solid gold nanoparticles are relatively low. In addition, prior art delivery systems are not able to modulate the timing of drug release (slow it down or accelerate it) because ordinary solid spherical particles do not efficiently convert the light energy into heat in the near-infrared region.

### SUMMARY OF THE INVENTION

A drug delivery system comprising a plurality of microspheres (“MS”) wherein each microsphere contains at least one drug product and a plurality of hollow gold nanospheres (“HAuNS”) is provided. The release of drug product (such as anticancer agents) is modulated by a photothermal effect mediated by a near-infrared (“NIR”) laser and the hollow gold nanospheres. Also, methods of drug entrapment for NIR light mediated drug release are provided herein. In a first method, positively charged or negatively charged drugs form a complex directly with hollow gold nanospheres (“HAuNS”) through electrostatic interaction. In a second method, hydrophilic (water-soluble) or hydrophobic (liposoluble) drugs are incorporated into polymeric matrix together with HAuNS.

### BRIEF DESCRIPTION OF THE DRAWINGS

FIG. 1A shows the absorption spectrum of HAuNS showing the plasmon resonance peak tuned to the NIR region (λmax=808 nm). FIG. 1B provides TEM images of HAuNS, revealing the morphology of the hollow nanospheres. Bar, 20 nm. FIG. 1C provides SEM images of PTX-loaded, HAuNS embedded microspheres (PTX/HAuNS-MS) and microspheres containing only PTX (PTX-MS). The presence of HAuNS in PTX/HAuNS-MS resulted in microspheres with smoother surface than PTX-MS without HAuNS. Bar, 10 μm. FIG. 1D are TEM photographs of a PTX/HAuNS-MS showing clusters of HAuNS dispersed within polymeric matrix.

FIG. 2A provides DSC thermograms of PTX/HAuNS-MS, pure PTX, and physical mixture of PLGA and PTX (5% PTX, w/w). An endothermic peak at 223° C. representing the melting point of PTX was present in the physical mixture of PTX and MS, but not in PTX/HAuNS-MS, suggesting that PTX was dissolved in PLGA polymer at the molecular level. FIG. 2B offers a comparison of temperature change of aqueous solutions containing HAuNS and HAuNS-MS after exposure to NIR light at an output power of 4.5 W/cm². HAuNS-loaded PLGA MS were able to elevate the temperature of water to the same extent as did HAuNS at a concentration of 4.2×10¹⁰ nanoparticles/mL.

FIG. 3A and FIG. 3B are the results of NIR light triggered release of PTX from PTX/HAuNS-MS including the 5-min period during which the microspheres were irradiated with NIR laser. FIG. 3A provides PTX release profiles from PTX/HAuNS-MS (formulation B) at NIR output power of 7 W (⋄), 4 W (*), and 2 W (∘) and PTX release profiles from PTX-MS (formulation E) at NIR output power of 7 W (Δ), and without NIR exposure (). FIG. 3B provides data of the PTX release from PTX/HAuNS-MS containing 4.7×10¹⁰ HAuNS/mg PLGA (formulation C) at NIR output power of 4 W (Δ) and 2 W (⋄) and PTX release from PTX/HAuNS-MS containing 9.4×10⁹ HAuNS/mg PLGA (formulation B) at NIR output power of 4 W (+) and 2 W (×), respectively. The spot size was 10 mm in diameter.

FIGS. 4A1, A2, B1, B2 and B3 show the cytotoxicity in the presence and absence of NIR irradiation. MDA-MB321 or U87 glioma cells were incubated with various MS formulations for 72 h. For NIR treatment, cells were irradiated with NIR laser 4 times at an output power of 2 W for 3 min each. Cells were incubated with PTX/HAuNS-MS, PTX-MS, and HAuNS-MS. Data is presented as mean plus/minus standard deviation of triplicate measurements. *P<0.01 compared to all the other treatment groups. #p<0.01 compared to all the other treatment groups except cells treated with HAuNS-MS.

FIG. 5A depicts antitumor activity of various treatments on U87 human glioma grown in nude mice. The microspheres were injected intratumorally in a single dose when tumor volume reached 100 mm³. Data are presented as mean±SD of tumor volumes (n=4-5).

FIG. 5B depicts antitumor effect of various treatments on MDA-MB-231 human breast carcinoma inoculated in mammary fatpats of nude mice. The drugs were injected intratumorally in a single dose when tumor volume measured 200 mm³. Data are presented as mean±SD of tumor volumes (n=5). Arrows indicate times at which tumors were removed for histologic analysis and (a-c) correspond to photographs a-c shown in FIG. 5C.

FIGS. 5C, 5D & 5E are microphotographs of tumor tissues removed at different times from mice treated with PTX/HAuNS-MS at a high dose of 6.0 mg equivalent PTX/kg (2.82×10¹⁰ HAuNS particles/mouse) plus laser irradiation. Tumors were burned initially, but gradually healed and became scars. No microscopic tumor cells were found in the scar tissues.

FIG. 6 depicts the hypothetical structure of PTX/HAuNS-MS and proposed mechanism of NIR-triggered drug release from the microspheres. PTX is dispersed uniformly in a polymeric matrix of polymer, while HAuNS are primarily dispersed in the inner water phase in the microspheres.

FIGS. 7A and 7B show the surface plasmon resonance absorption of hollow gold nanospheres (HAuNS) embedded in PLGA microspheres (MS) mediated controllable release of paclitaxel (PTX) from the microspheres upon near-infrared light irradiation.

FIGS. 8A and 8B are illustrative of the hollow gold nanospheres described herein with a strong surface plasmon absorption in the near-infrared region that forms stable complexes with drugs such as doxorubicin and with extremely high payload. The drug (DOX as shown) is released intracellularly upon near-infrared light irradiation.

FIGS. 9A, 9B, 9C, 9D1 and 9D2 show generally the fabrication and characterization of DOX-loaded hollow gold nanospheres (DOX@HAuNS). Specifically, FIG. 9A is a schematic of HAuNS synthesis and TEM images of plain and DOX-loaded HAuNS (DOX@HAuNS and DOX@PEG-HAuNS). FIG. 9B is absorption spectra of HAuNS and PEG-HAuNS. FIG. 9C shows absorbance spectra of free DOX, HAuNS, and DOX@HAuNS. FIG. 9D1 and 9D2 show absorbance spectra of initial physical mixture of DOX and HAuNS (purple) and the complexes of DOX and HAuNS after 24 h of incubation. Significant fluorescence quenching was observed for DOX@HAuNS.

FIGS. 10A, 10B1, 10B2, 10C and 10D show Langmuir adsorption isotherms showing the adsorption of DOX by HAuNS and PEG-HAuNS with various PEG:Au molar ratios. The absorbed DOX was plotted against the initial amount of DOX in the solution. FIGS. 10B1 and 10B2 show a comparison of DOX payload (FIG. 10B1) and particle size (FIG. 10B2) between DOX@HAuNS and DOX@PEG-HAuNS at the maximal DOX loading. Low PEG density increased DOX payload. PEGylation stabilized DOX-loaded HAuNS. Particle size was measured by dynamic light scattering after a mild water-bath ultrasound treatment. FIG. 10C shows the stability of DOX@HAuNS and DOX@PEG-HAuNS in various media.

FIGS. 11A, 11B, 11C and 11D depict NIR light triggered release of DOX from DOX@HAuNS and DOX@PEG-HAuNS. FIG. 11A shows DOX release profiles in the presence and absence of NIR laser. Irradiation with NIR laser caused rapid DOX release during NIR exposure (5 min, red lines), and the release was turned off when laser was switched off. FIG. 11B shows absorption spectra of DOX@HAuNS before and after NIR laser irradiation. Aqueous solutions of (a) DOX@HAuNS before laser irradiation, (b) released DOX collected in the supernatant, and (c) DOX@HAuNS after complete release of DOX are shown. FIG. 11C shows the effect of pH on DOX retention on PEG-HAuNS. FIG. 11D is a comparison of NIR light-triggered release of DOX at different pH.

FIGS. 12A and 12B show the uptake of DOX@HAuNS and free DOX in MDA-MB-231 breast cancer cells. FIG. 12 A shows the cellular uptake of DOX@HAuNS after 1 h and 48 h of incubation. The reflectance of HAuNS was visualized using a dark-field condenser. The red color from the fluorescence signal of DOX@HAuNS was localized in spots in the cytoplasm. FIG. 12B shows the cellular uptake of free DOX and DOX@HAuNS treated with NIR laser (1.0 W/cm² for 3 min per treatment, 4 treatments over 2 h, incubated for 8 h). Cell nuclei were counterstained with DAPI. The red fluorescence signal from DOX in DOX@HAuNS was localized to the cell nuclei after NIR laser irradiation, indicating intracellular release of DOX from DOX@HAuNS upon NIR exposure.

FIGS. 13A and 13B show cell survival as a function of DOX concentration (FIG. 13 A) and Au concentration (FIG. 13B). MDA-MB-231 cells were either not exposed to NIR light or irradiated with NIR light (2 W/cm² for 3 min per treatment, 4 treatments over 2 h). The viability of cells was determined using the MTT assay. Data represent mean±standard deviation of triplicate experiments.

FIGS. 14A and 14B are the TEM images of DOX@HAuNS before and after NIR laser irradiation. Irradiation with NIR light caused fragmentation of a small fraction of HAuNS particles (arrows).

FIGS. 15A, 15B, 15C and 15D show the comparison between hollow gold nanospheres (HAuNS) and solid gold nanoparticles (AuNP). FIG. 15A is a comparison of DOX payload between HAuNS and solid AuNP. FIG. 15B is a release of DOX from DOX@HAuNS (blue line) and DOX@AuNP (pink line) under repeated NIR laser exposure. FIG. 15C is a comparison of absorption spectra among AuNP, DOX@AuNP, and DOX@HAuNS. FIG. 15D is the temperature change measured over a period of 10 min of exposure to NIR light at an output power of 5.0 W/cm². All nanoparticles had the same concentration of 0.7×10¹¹ particles/mL.

FIGS. 16A and 16B shows the mechanism of entrapment for (A) positively charged drugs (i.e. Dox) and (B) negatively charged drugs with HAuNS.

FIG. 17A and FIG. 17B show the likely mechanism of NIR light-induced drug release for liposoluble drug (i.e. PTX) (FIG. 17A) and hydrophilic drug (i.e. Dox) from PLGA microspheres (FIG. 17B).

FIG. 18 shows fluorescent microphotographs of PLGA microspheres loaded with Dox and HAuNS.

### DETAILED DESCRIPTION

Presented herein are drug delivery systems that comprise a plurality of biodegradable and biocompatible microspheres (“MS”) wherein each microsphere contains at least one drug product and a plurality of hollow gold nanospheres (“HAuNS”). The diameter of the microspheres can be between about 100 nm to 200 nm. The HAuNS diameter can be between about 20 nm to about 80 nm, and the thickness of the shell of the hollow gold nanosphere can be between about 2 nm to about 8 nm. The plasma resonance absorption peak can be tuned by controlling the diameter and the thickness of the gold shell of HAuNSs. The diameter and thickness of the HAuNSs can be controlled by varying the size of the cobalt nanoparticles (which serve as a template), the ratio of cobalt nanoparticles to chloroauric acid, the removal of oxygen from the reaction mixture, and the rate of stirring of the reaction mixture. The diameter of the microspheres can be controlled by controlling the ratio of organic solvent to aqueous solution, the rate of stirring, the selection of surfactant, the molecular weight and concentration of the polymers used, among other parameters.

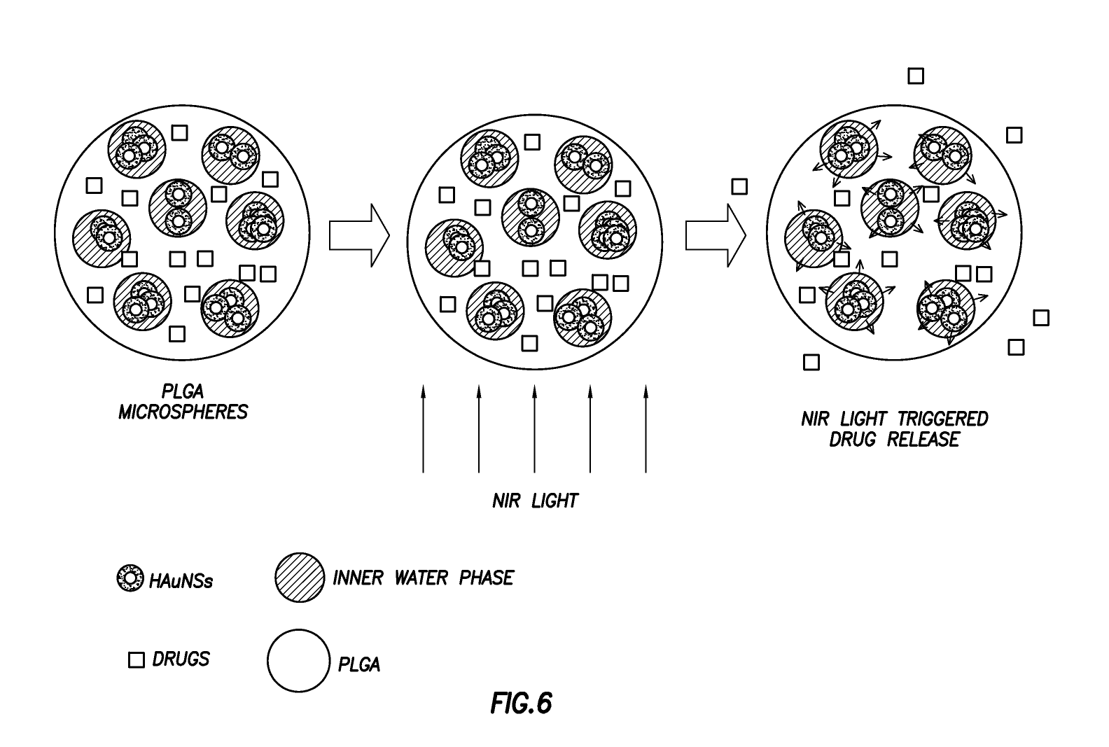

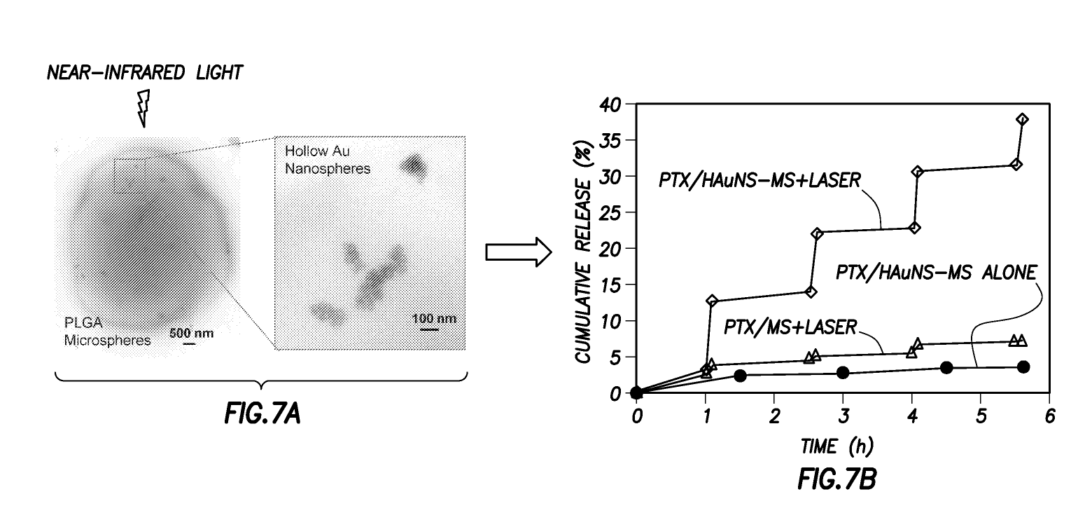

As shown in FIG. 6, the microspheres can contain both the drug (also sometimes referred to herein as a therapeutic agent or drug product) and the HAuNS as the photothermal coupling agent. The microspheres are mediated by NIR light and as described below have been evaluated for the drug release properties including in vitro cytotoxicity, and in vivo antitumor activity. The HAuNS display surface plasmon absorbance in the near-infrared (NIR) region. The photothermal effect mediated by a near-infrared (NIR) laser and hollow gold nanospheres (HAuNS) modulates the release of anticancer agents.

The microspheres (also referred to sometimes as “MS”) can be made of polymeric material and are sometimes fabricated with biodegradable, biocompatible poly(lactide-co-glycolide) copolymers (referred to sometimes as “PLGA”). Other biodegradable polymeric materials that may be used as polymeric material for preparation of microspheres include, but are not limited to, Poly(lactic acid), poly(glycolic acid), poly(dioxanone), poly(trimethylene carbonate), poly(ε-caprolactone) homopolymers and their copolymers, polyanhydrides, polyorthoesters, polyphosphazenes, and the like.

The HAuNS-containing microspheres presented herein have an NIR light-induced thermal effect similar to that of plain HAuNS. (FIG. 6). Specifically, when microspheres are irradiated with NIR light, drugs can be rapidly and repetitively released from the microspheres containing the drug and HAuNS. Drug release is insignificant when NIR light is switched off. The release of the drug from the microspheres can be readily controlled by NIR laser output power, duration of laser irradiation, treatment frequency, and the concentration of HAuNS embedded inside the microspheres.

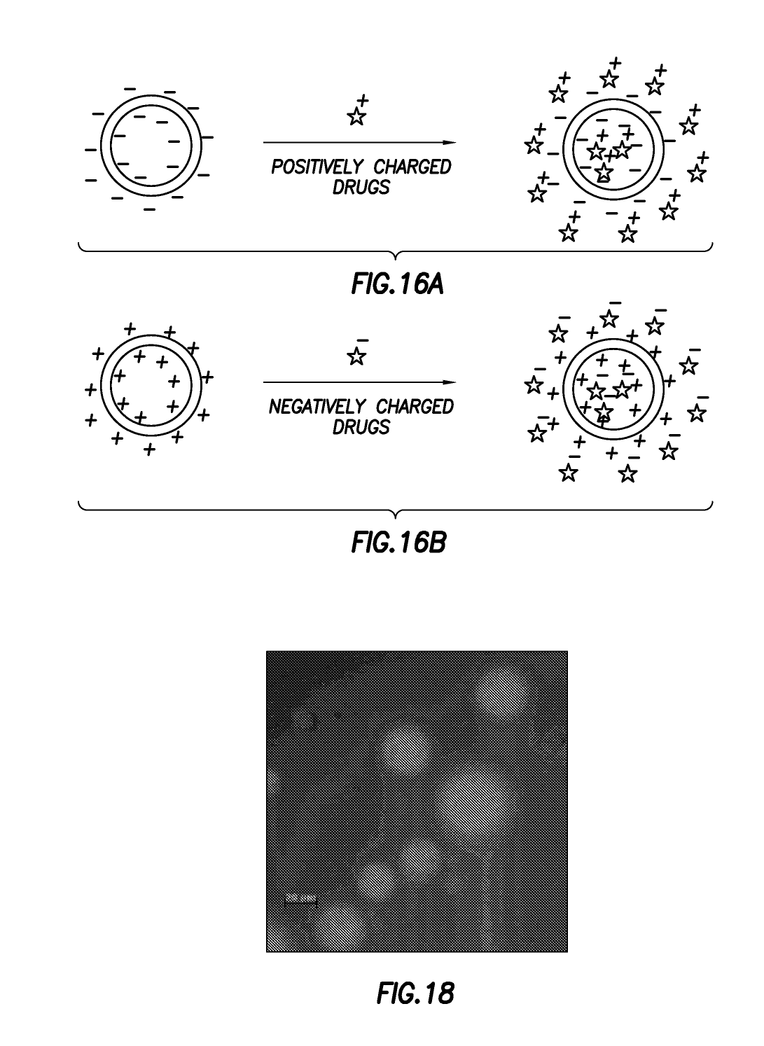

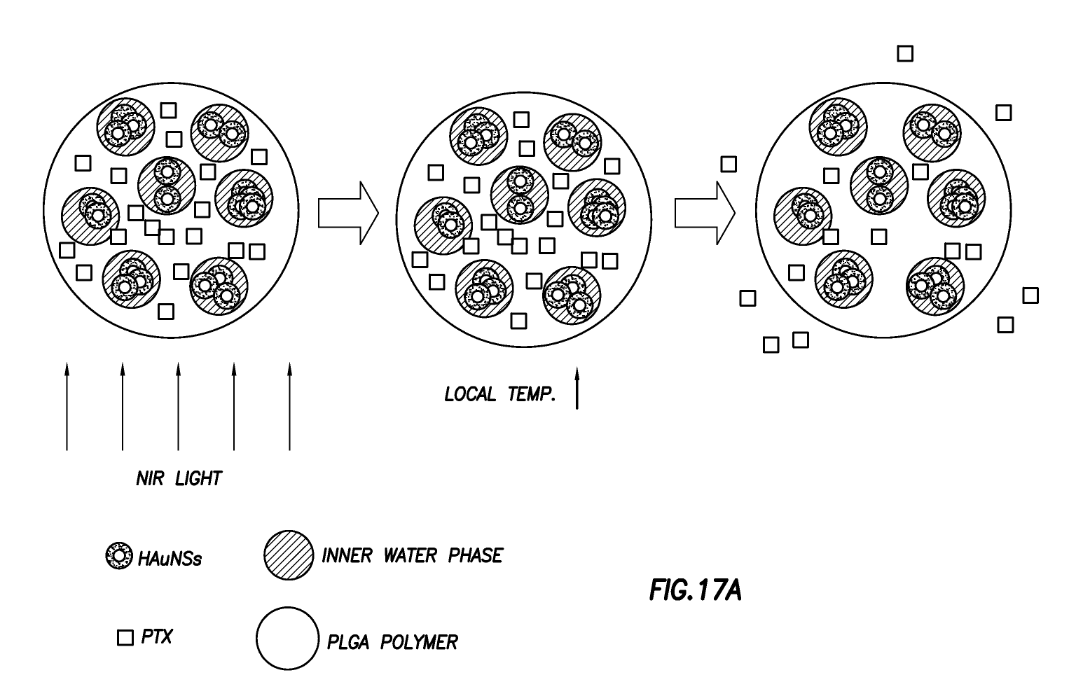

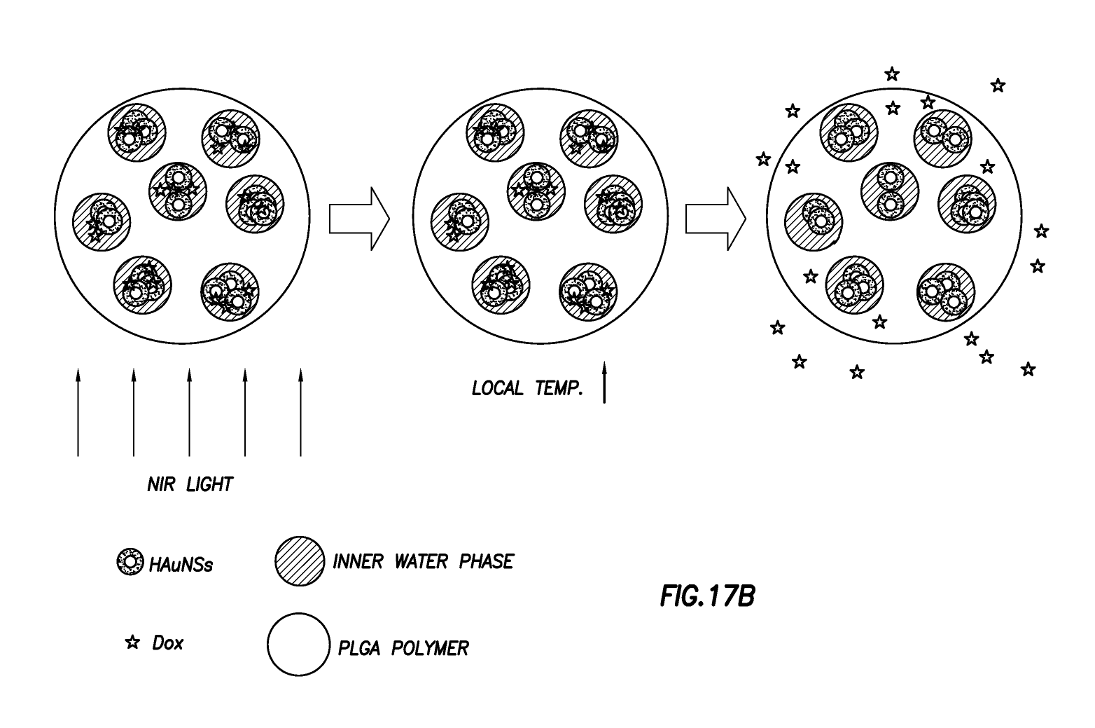

Also, provided herein are two methods of drug entrapment for NIR light mediated drug release. In a first method, positively charged or negatively charged drugs are complexed directly with hollow gold nanospheres (HAuNS) through electrostatic interaction. In a second method, hydrophilic (water-soluble) or hydrophobic (liposoluble) drugs are incorporated into polymeric matrix together with HAuNS. FIG. 16 depicts the mechanism for the entrapment of charged drugs to HAuNS. FIG. 17 depicts entrapment of liposoluble and water-soluble drugs in polymeric nanoparticles/microparticles and the likely mechanism for NIR light-induced drug release for liposoluble drugs (i.e., paclitaxel (“PTX”)) and water-soluble drugs (i.e., doxorubicin “Dox”)) from PLGA microspheres. In FIG. 17A, PTX is dispersed uniformly in the polymeric matrix of PLGA polymer, while HAuNS are primarily dispersed in the inner water phase in the microspheres. In FIG. 17B, Dox and HAuNS are all dispersed in the inner water phase. Upon NIR irradiation, HAuNS produce a strong photothermal effect, rapidly raising the temperature of the microenvironment in the inner water droplets and the microspheres. For PTX dispersed in the polymeric matrix, increased local temperature leads to increases in the diffusion coefficient of the drug in the polymeric matrix and the flexibility of polymer chains, resulting in an accelerated drug release. A similar mechanism occurs for the release of Dox in the inner water phases in the microspheres. FIG. 18 shows fluorescent microphotographs of PLGA micospheres containing Dox and HAuNS prepared using the double emulsion technique. Red fluorescence indicates the presence of Dox inside the microspheres

Positively charged drugs useful in connection with the drug delivery system and methods presented herein include, but are not limited to Doxorubicin hydrochloride, Daunorubicine hydrochloride, Cisplatin, Gemcitabine, Cytarabine, Mitoxantrone hydrochloride, Vincristine sulfate, Vinblastine sulfate, Bleomycin sulfate, Irinotecan hydrochloride, Topotecan hydrochloride, Cefotiam, Lamivudine, tetracycline hydrochloride, Moxifloxacin hydrochloride, and the like.

Negatively charged drugs useful in connection with the drug delivery system and methods presented herein include, but are not limited to, Methotrexate sodium, Porfilmer sodium, Sefonicid sodium, Amphotericin B, Hydrocortisone sodium succinate, Sulfacetamide sodium, Cefminox Sodium, Prostaglandin E1, Ampicillin sodium, Ticarcillin disodium, and the like.

Representative liposoluble drugs that may be used in connection with the methods and drug delivery system described herein include, but are not limited to, Paclitaxel, Docetaxel, Tamosifen, Melphalan, Etopside, Bortezomib, camptothecin, Cyclopamine, Carboplatin, Oxaliplatin, Imatinib, Cyclosporin, Geldanamycin, 17-(allylamino)-17-demethoxygeldanamycin, Rapamycin, Valsartan, Simvastatin, Olanzapine, and the like.

Water-soluble drugs that may be used in connection with the drug delivery system and methods described herein include, but are not limited to Daunorubicine hydrochloride, Cisplatin, Gemcitabine, Cytarabine, Mitoxantrone hydrochloride, Vincristine sulfate, Vinblastine sulfate, Bleomycin sulfate, Irinotecan hydrochloride, Topotecan hydrochloride, Cinepazide, Maleate, Acarbose, Clopidogrel, Azithromycin, and the like.

In addition, the drug delivery system may be used in connection with protein/peptide drugs that can be loaded together with HAuNS into the polymeric matrix. The protein/peptide drug products include, but are not limited to, Thymotin, insulin, and the like. Importantly, water-soluble MRI contrast agents such as DTPA-Gd can be loaded into polymeric matrix together with HAuNS and therapeutic agents to help monitor the distribution and release of drugs from the polymeric nanoparticles/microparticles upon NIR laser irradiation. Combinatorial drug delivery can be achieved by loading more than one drug to HAuNS or HAuNS-loaded microspheres.

Drug delivery strategies are generally directed to increasing the drug concentration at the target location, decreasing systemic toxicity, and allowing greater temporal control over drug release compared to traditional drug delivery systems. Controlled drug delivery occurs when a polymer, whether natural or synthetic, is combined with a drug or other active agent in such a way that the active agent is released from the material in a predetermined manner. The release of the active agent may be constant over a long period. It may be cyclic, or it may be triggered by the environment or other external events. The main purpose behind controlling drug delivery is to achieve more effective therapies while eliminating the potential for both under- and overdosing. A major challenge for drug delivery is to control drug release both spatially and temporally.

Drug delivery triggered by internal or external stimuli, such as pH, enzymes, magnetism, ultrasound, and heat, has recently attracted much attention. See e.g., Lee, E. S. et al., Nanomed. 2008, 3, 31-43; Lai, C. Y. et al., 125, 4451-4459, J. Am. Chem. Soc. 2003; P. Meers, 53, 265-272, Adv. Drug Deliver. Rev. 2001; Ankareddi, I. et al., 2, 431-434, Nanotech. 2007; Derfus, A. et al., 19, 3932-3936, Adv. Mater. 2007; De Geest, B. et al., 3, 804-808, Small 2007; Deckers, D. et al., 60, 1153-1166, Adv. Drug Deliver. Rev. 2008; Kim, H. J. et al., 18, 3083-3088, Adv. Mater. 2006; Needham, D. et al., 53, 285-305, Adv. Drug Deliver. Rev. 2001.

Light-triggered drug release is one approach for drug delivery. These spatiotemporal controlled delivery systems are responsive to light on the basis of photochemical mechanisms. See, Zhang, Z. Y., et al., 10, 1150-1152, Bioconjug. Chem. (1999); Wijtmans, M. et al., J., 128, 11720-11726, Am. Chem. Soc. (2006); Alvarez-Lorenzo, C. et al., 85, 848-860 Photochem. Photobiol. (2009). These systems are limited to in vitro use because ultraviolet/visible light used to activate the release of drugs cannot penetrate skin.

Conversely, a photothermal phenomenon mediated by gold nanoparticles is used by the drug delivery system described herein. Gold nanostructures such as gold nanoshells, hollow gold nanospheres (HAuNS), and gold nanorods can be used in photothermal ablation of cancer cells. See e.g., Hirsch, et al., 100, 13549-13554, Proc. Natl. Acad. Sci. USA (2003); Loo, et al., 3, 33-40, Technol. Cancer Res. Treat. (2004); W. Lu, et al., 15, 876-886, Clin. Cancer Res. (2009); Melancon, et al., 7, 1730-1739, Mol. Cancer. Ther. (2008); Huang, et al., 128, 2115-2120, J. Am. Chem. Soc. (2006); Takahashi, et al., 35, 500-501, Chem Lett (2006).

Noble metal nanoparticles exhibit a strong optical extinction at near-infrared (NIR) wavelengths (700-850 nm) owing to the localized surface plasmon resonance of their free electrons upon excitation by an electromagnetic field. Absorption of NIR light results in resonance and the transfer of thermal energies to the surrounding medium or tissue. NIR light (700-850 nm) can readily penetrate skin and go deep into the tissue because tissue absorption of light in the NIR region is minimal. R. Weissleder, 19, 316-317, Nat. Biotechnol. (2009). For example, SiO₂—Au nanoshells embedded within temperature-sensitive hydrogels demonstrate modulated drug delivery profiles. M. Bikram, et al., 123, 219-227, J. Controlled Rel. (2007). However, this system has not yet been translated to injectable colloidal delivery vehicles suitable for in vivo applications. Similarly, fabricated multi-layered polyelectrolyte microshells containing aggregates of colloidal gold have been used in NIR-triggered release of dextran. Bedard, et al., 2, 1807-1816, ACS Nano (2008). In addition, a femtosecond pulsed laser to trigger release of a dye molecule from liposomes containing HAuNS has been shown. G. Wu, et al., 130, 8175-8177, J. Am. Chem. Soc. (2008). However, the release of therapeutically significant anticancer drugs has not previously been shown nor has an in vivo evaluation of these delivery systems been performed.

Hollow gold nanospheres are a class of gold nanoparticles having plasmon absorption in the NIR region that display strong photothermal coupling property suitable for photothermal ablation (PTA) therapy. The unique combination of small size (30-50 nm in diameter), absence of a silica core, spherical shape, strong and tunable (520-950 nm) absorption band, and the lack of need for cytotoxic surfactant required to stabilize other gold nanoparticles make HAuNS a useful alternative as an in vivo molecular therapy.

A gold nanoparticle (“AuNP”) generally provides unique chemical and physical properties, including their functional versatility, biocompatibility, and low toxicity. Templeton, A. C. et al., Monolayer-Protected Cluster Molecules, 33 (1), 27-36, Acc Chem Res (2000); De, M. et al., Applications of Nanoparticles in Biology, 20 (22), 4225-4241, Adv Mater (2008); Bhattacharya, R. et al., Biological Properties Of “Naked” Metal Nanoparticles, 60 (11), 1289-306, Adv Drug Deliv Rev (2008); Connor, E. E. et al., Gold Nanoparticles Are Taken Up By Human Cells But Do Not Cause Acute Cytotoxicity, 1 (3), 325-7, Small (2005).

Furthermore, gold nanoparticles will absorb light in the near infrared, a spectral region that is barely absorbed by tissue. The absorbed light energy causes the gold particles to vibrate and is dissipated into the surrounding area as heat. The tiny gold particles can be functionalized so that the specifically bind to tumor cells. Thus, only cells that contain gold particles are killed off.

While gold nanoparticles (“AuNPs”) currently used for biosensing and diagnostics, gold nanoparticles have also been investigated as potential nanocarriers for delivery of various drugs into their targets. Cheng, M. M. et al., Nanotechnologies for Biomolecular Detection and Medical Diagnostics, 10 (1), 11-9, Curr Opin Chem Biol (2006); Rosi, N. L. et al., Nanostructures in Biodiagnostics, 105 (4), 1547-62, Chem Rev (2005); Baptista, P. et al., Gold Nanoparticles for the Development of Clinical Diagnosis Methods, 391 (3), 943-50, Anal Bioanal Chem (2008). The payloads of AuNPs may include small drug molecules or large biomolecules, such as proteins, DNA, or RNA. Gibson, J. D. et al., Paclitaxel-Functionalized Gold Nanoparticles, 129 (37), 11653-61, J Am Chem Soc (2007); Cheng, Y. et al., Highly Efficient Drug Delivery With Gold Nanoparticle Vectors For In Vivo Photodynamic Therapy of Cancer, 130 (32), 10643-7, J Am Chem Soc (2008); Kim, C. K. et al., Entrapment Of Hydrophobic Drugs In Nanoparticle Monolayers With Efficient Release Into Cancer Cells, 131(4), 1360-1, J Am Chem Soc (2009); Chithrani, B. D. et al., Elucidating The Mechanism Of Cellular Uptake And Removal Of Protein-Coated Gold Nanoparticles Of Different Sizes And Shapes, 7 (6), 1542-50, Nano Lett (2007); Visaria, R. K. et al., Enhancement Of Tumor Thermal Therapy Using Gold Nanoparticle Assisted Tumor Necrosis Factor-Alpha Delivery, 5 (4), 1014-20, Mol. Cancer. Ther. (2006); Ghosh, P. S. et al., Efficient Gene Delivery Vectors By Tuning The Surface Charge Density Of Amino Acid-Functionalized Gold Nanoparticles, 2 (11), 2213-8, ACS Nano (2008); Lee, J. S. et al., Gold, Poly(Beta-Amino Ester) Nanoparticles For Small Interfering RNA Delivery, 9 (6), 2402-6, Nano Lett (2009); Elbakry, A. et al., Layer-By-Layer Assembled Gold Nanoparticles for siRNA Delivery, 9 (5), 2059-64, Nano Lett, (2009).

Further, the use of gold nanoparticles as drug carriers for doxorubicin (DOX) delivery and have shown enhanced cytotoxic effects with this approach in vitro. Dhar, S. et al., Natural Gum Reduced/Stabilized Gold Nanoparticles For Drug Delivery Formulations, 14(33), 10244-50, Chemistry (2008); Kumar, S. A. et al., Facile Biosynthesis, Separation And Conjugation Of Gold Nanoparticles To Doxorubicin, 19 (49), Nanotechnology (2008).

Indeed, efficient active targeting of HAuNS and selective PTA of solid tumors in small animals using HAuNS have been directed at epidermal growth factor receptor (EGFR) and melanocortin type-1 receptor overexpressed in melanoma. Melancon, M. P., et al., In Vitro And In Vivo Targeting Of Hollow Gold Nanoshells Directed At Epidermal Growth Factor Receptor For Photothermal Ablation Therapy, 7 (6), 1730-1739; Mol Cancer Ther (2008); Lu, W., et al., Targeted Photothermal Ablation Of Murine Melanomas With Melanocyte-Stimulating Hormone Analog-Conjugated Hollow Gold Nanospheres, 15 (3), 876-86, Clin Cancer Res (2009). These HAuNS exhibited excellent colloidal stability, enhanced tumor uptake, and efficient PTA effect against human tumor xenografts after intravenous injection. Id.

On the other hand, although NIR light can penetrate several centimeters of tissues, its energy is gradually reduced as it travels deeper into tissues due to light scattering and absorption. Some tumor cells would inevitably receive sub-optimal laser exposure and may not be ablated. We hypothesized that the unique photothermal conversion property of HAuNS could be harnessed for modulating the delivery of anticancer drugs, thus making it possible to achieve significantly enhanced antitumor efficacy using two-punch approach in a single setting. In the work described below, we investigated the utility of using HAuNS as a nanocarrier for DOX, and the potential application of DOX-loaded HAuNS (DOX@HAuNS) for anticancer therapy.

HAuNS as a Nanocarrier for Drug Delivery.

This approach is advantageous in several aspects. First, HAuNS displayed exceptionally high drug loading capacity and stability (as exemplified by studies of DOX delivery) owing to the unique physicochemical characteristics of HAuNS. In particular, the hollow interior of the nanoparticles allowed significant increase in effective surface area for DOX attachment, resulting in 3.5-fold increase in DOX payload compared with solid AuNP of the same size, surface charge, and weight. Second, HAuNS mediated strong photothermal effect owing to their strong surface plasmon absorption in the NIR region. This property was exploited for controlled release of DOX from DOX@HAuNS using NIR light as the external stimulus to trigger drug release. Third, dual models of cell killing were integrated into a single nanodevice, i.e., photothermal ablation mediated by HAuNS and antitumor activity of DOX released from HAuNS upon NIR laser irradiation. This “two-punch” approach is expected to significantly increase the likelihood of cell killing and potentially overcome resistance to chemotherapeutic agents, making it a promising approach to cancer therapy.

As described herein below, in vitro, cancer cells incubated with microspheres loaded with paxlitaxel (“PTX”) and hollow gold nanospheres (“HAuNS”) were irradiated with NIR light displayed showed significantly greater cytotoxic effects than cells incubated with the microspheres alone or cells irradiated with NIR light alone, owing to NIR light-triggered drug release. Specifically as described herein, the treatment of human U87 gliomas and MDA-MB-231 mammary tumor xenografts in nude mice with intratumoral injection of these PTX/HAuNS-loaded microspheres followed by NIR irradiation resulted in significant tumor growth delay compared to tumors treated with HAuNS-loaded microspheres (no PTX) and NIR irradiation or with PTX/HAuNS-loaded microspheres alone. As a result, a novel therapeutic approach is herein provided where NIR light can be used for simultaneous modulation of drug release, and induction of photothermal cell killing.

### Example I

### Experimental

### Reagents

PLGA (lactide:glycolide=50:50, viscosity=0.20 dl/g) was purchased from DURECT Corp. (Cupertino, Calif.). Polyvinyl alcohol (PVA; MW˜25000, 88 mol % hydrolyzed) was purchased from Polysciences, Inc. (Warrington, Pa.). PTX was provided by Yunnan Hande Bio-Tech Co., Ltd. (Houston, Tex.). Trisodium citrate dehydrate (>99%), cobalt chloride hexahydrate (99.99%), sodium borohydride (99%), and chloroauric acid trihydrate (ACS reagent grade) were purchased from Fisher Scientific (Pittsburgh, Pa.) and were used as received. 3-(4,5-Dimethylthiazol-2-yl)-2,5-diphenyltetrazolium bromide (MTT) and Tween 80 were purchased from Sigma-Aldrich (St. Louis, Mo.). Methylene chloride was obtained from Baxter Healthcare Corp. (Deerfield, Ill.).

### Cell Lines

U87 (human glioma) and MDA-MB-231 (human breast carcinoma) cell lines were obtained from American Type Culture Collection (Manassas, Va.). Cells were maintained at 37° C. in a humidified atmosphere containing 5% CO₂ in Dulbecco's modified Eagle's medium/nutrient mixture F-12 Ham and 10% fetal bovine serum (Life Technologies, Inc., Grand Island, N.Y.).

### Synthesis and Characterization of HAuNS

HAuNS were synthesized according to previously reported procedures. See e.g., W. Lu, et al., 15, 876-886, Clin. Cancer Res. (2009); M. P. Melancon, 7, 1730-1739, Mol. Cancer. Ther. (2008). Briefly, cobalt nanoparticles were first synthesized by reducing cobalt chloride (1 mL, 0.4 mol/L) with sodium borohydride (4.5 mL, 1 mol/L) in deionized water containing 2.8 mL of sodium citrate (0.1 mol/L). HAuNS were obtained by adding chloroauric acid into the solution containing cobalt nanoparticles. The size of the HAuNS was determined using dynamic light scattering on a Brookhaven particle size analyzer (Holtsville, N.Y.). Ultraviolet-visible spectroscopy was recorded on a Beckman Coulter spectrometer (Fullerton, Calif.). The morphology of HAuNS was examined with a JEM 1010 transmission electron microscope (TEM) (Peabody, Mass.). The concentration of the nanoparticles was estimated based on absorbance at 808 nm and an extinction coefficient of ε=8.3×10⁹ L/mol/cm (1.37×10⁻¹¹ mL/particle/cm).

### Preparation and Characterization of PLGA Microspheres

A modified water-in-oil-in-water (W1/O/W2) double-emulsion solvent evaporation method was employed to prepare PLGA microspheres containing PTX and HAuNS (PTX/HAuNS-MS). The first emulsion was formed by mixing an aqueous solution (0.08 mL) containing HAuNS with dichloromethane (0.8 mL) containing PLGA (240 mg) and PTX (12.6 mg), which was then injected into an aqueous solution of polyvinyl alcohol (2% PVA, 8.0 mL) serving as the external aqueous phase. W1/O/W2 emulsion was achieved using a POLYTRON PT-MR 3000 benchtop homogenizer from Kinematica AG (Lucerne, Switzerland) at 15,000 rpm. The microspheres were formed after the organic solvent was completely evaporated, washed three times with water, and freeze-dried. Microspheres containing only PTX (PTX-MS) and microspheres containing only HAuNS (HAuNS-MS) were also prepared using the same procedures.

The size and morphology of the microspheres were examined with a JSM-5910 scanning electron microscope (JEOL, USA, Inc., Peabody, Mass.). Differential scanning calorimetry (DSC) was performed on a Q2000 system from TA Instruments (New Castle, Del.). Samples of ˜5 mg were first held isothermal at −20° C. for 5 minutes and then heated to 300° C. at a rate of 20° C./min. All DSC tests used aluminum pans and 50 mL/min of N₂ purge.

For measuring the photothermal effect of the HAuNS-containing microspheres, 808-nm NIR laser light was delivered through a quartz cuvette containing the HAuNS or HAuNS-containing microspheres (100 μL). A thermocouple was inserted into the solution perpendicular to the path of the laser light. The temperature was measured over a period of 15 min. Phosphate-buffered saline was used as a control. The laser was a continuous-wave GCSLX-05-1600m-1 fiber-coupled diode laser (China Daheng Group, Beijing, China). A 5-m, 600-μm core BioTex LCM-001 optical fiber (Houston, Tex.) was used to transfer laser light from the laser unit to the target. This fiber had a lens mounting at the output that allowed the laser spot size to be changed by changing the distance from the output to the target. The output power was independently calibrated using a handheld optical power meter (model 840-C; Newport, Irvine, Calif.) and was found to be 1.5 W for a spot diameter of 6.5 mm (˜4.5 W/cm²) and a 2-amp supply current.

### PTX and HAuNS Loading and Encapsulation Efficiency

The amount of PTX in the microspheres was determined by an Agilent 1100 Series high-performance liquid chromatography (HPLC) (Santa Clara, Calif.), used according to the reported procedures J. You, et al., 8, 2450-2456, Biomacromol. (2007). PTX loading was expressed as PTX content in dry microspheres (w/w). The entrapment efficiency (EE) was expressed as the percentage of the actual PTX loading over the theoretical PTX loading. The loading efficiency for HAuNS was determined by measuring the absorbance of the microspheres at 808 nm. A standard curve was constructed with known concentrations of HAuNS in aqueous solution.

NIR Light-Triggered Release of PTX from PLGA Microspheres

The release studies were performed at room temperature. A solution of PTX/HAuNS-MS (20 mg) in PBS (0.01 M, 2.0 mL, pH 7.4) containing Tween-80 (0.1%, w/v) was placed in a test tube. A laser probe (10 mm spot diameter) was fixed 5 cm from the center of the test tube. The samples were irradiated with the 808-nm NIR light at an output power of 2-10 W over a period of 5 min (Diomed 15 plus, Cambridge, UK). At various times, aliquots were drawn from the test tube and centrifuged at 5000 rpm for 5 min, and the released free PTX was quantified by HPLC.

### In Vitro Cytotoxicity

Cells (1.0×10⁴) were seeded in 96-well plates and incubated for 24 h to allow the cells to attach to the surface of the wells. The cells were then exposed to 0.01-10.0 mg/mL of PTX/HAuNS-MS, HAuNS-MS, or PTX-MS. The microsphere-treated cells were irradiated 4 times with NIR light at an output power of 2 W (3-min duration each time, 1-h intervals between irradiations) and a spot diameter of 10 mm. Microsphere-treated cells not irradiated with NIR laser were used as a control. All cells were incubated at 37° C. for 72 h. Cell survival was determined using the MTT cytotoxicity assay according to the manufacturer-recommended procedures. The data are presented as the mean±standard deviation of triplicate measurements.

To evaluate the relative contribution of the cytotoxic effect of PTX and photothermal effect of the microspheres, the cytotoxic activity of HAuNS-MS (without drug), PTX/HAuNS-MS, culture medium containing PTX/HAuNS-MS pre-treated with laser in the absence of cells, and PTX/HAuNS-MS treated with laser in the presence of MDA-MB-231 cells were compared. The concentrations of microspheres were 0.02, 1.0, 2.0, and 8.0 mg/mL. NIR light was delivered at an output power of 2 W and a spot diameter of 10 mm (2.55 W/cm²) for a period 5 min. Cell survival was determined using the MTT assay as described before. The PTX concentration in the medium after laser irradiation and after 72 h of incubation was measured by HPLC.

### In Vivo Antitumor Activity

All experiments involving animals were performed in accordance with the guidelines of the Institutional Animal Care and Use Committee. Female nude mice (nu/nu; 18-22 g; 6-8 weeks of age; Harlan, Indianapolis, Ind.) were anesthetized with isoflurane inhalation and inoculated subcutaneously with U87 cells (5×10⁶ cells in 0.1 ml PBS). When the tumor volume reached about 100 mm³, the mice were randomly allocated into four groups (n=4-5). Groups 1 and 2 received 20-μL intratumoral injections of PTX/HAuNS-MS (PTX: 1.0 mg/kg; 4.7×10⁹ HAuNS particles/mouse; formulation A). Group 3 received 20-4 intratumoral injections of HAuNS-MS (4.7×10⁹ HAuNS particles/mouse; formulation D). Group 4 received 20-μL intratumoral injections of saline. Tumors in mice from groups 1, 3, and 4 were irradiated with NIR light at an output power of 1.5 W for 5 min (1 treatment/day, total of four treatments for each tumor). For NIR treatment, the laser probe (10 mm spot diameter) was fixed 4 cm from the tumors. Tumor growth was determined 2-3 times a week by measuring two orthogonal tumor diameters. Tumor volume was calculated according to the formula (a×b²)/2, where a and b are the long and short diameters of a tumor, respectively. The effect of treatment on tumor growth was expressed as the growth delay, defined as the time in days for tumors in the treated groups to grow from 100 mm³ to 500 mm³.

Antitumor efficacy was further investigated in female nude mice bearing tumors from human breast cancer MDA-MB-231 cells (5×10⁶ cells in 0.1 ml PBS) inoculated orthotopically in the mammary fat pads. When the tumor volume reached about 200 mm³, the mice were randomly allocated into five groups (n=5). Mice in groups 1-3 received intratumoral injections of PTX/HAuNS-MS (formulation A) at an equivalent PTX dose of 1.0 mg/kg (4.7×10⁹ HAuNS particles/mouse, low dose), 6.0 mg/kg (2.82×10¹⁰ HAuNS particles/mouse, high dose), and 6.0 mg/kg (2.82×10¹⁰ HAuNS particles/mouse, high dose), respectively. Group 4 received intratumoral injections of HAuNS-MS (formulation D) at a dose of 4.7×10⁹ HAuNS particles/mouse (low dose). Group 5 received intratumoral injections of saline. The tumors in groups 1, 2, and 4 were also irradiated with the NIR laser at an output power of 1.5 W for 5 min (2 laser treatments/day for 4 consecutive days; total 8 treatments). Tumor growth was measured weekly, using the same protocol as described above. Tumor growth delay was defined as the time in days for tumors in the treated groups to grow from 200 mm³ to 1000 mm³.

For histologic evaluation, tumors were removed and cryosectioned for hematoxylin and eosin staining. The slices were examined under a Zeiss Axio Observer.Z1 microscope equipped with a Zeiss AxioCam MRc5 color camera (Thornwood, N.Y.).

### Data Analysis

Mean differences in the tumor growth delay (number of days required to grow from 100 mm³ to 500 mm³ for U87 tumors or from 200 mm³ to 1000 mm³ for MDA-MB-231 tumors) were analyzed by Student's t test, with P<0.05 considered to be statistically significant.

### Synthesis and Characterization of HAuNS and PTX-Loaded PLGA Microspheres

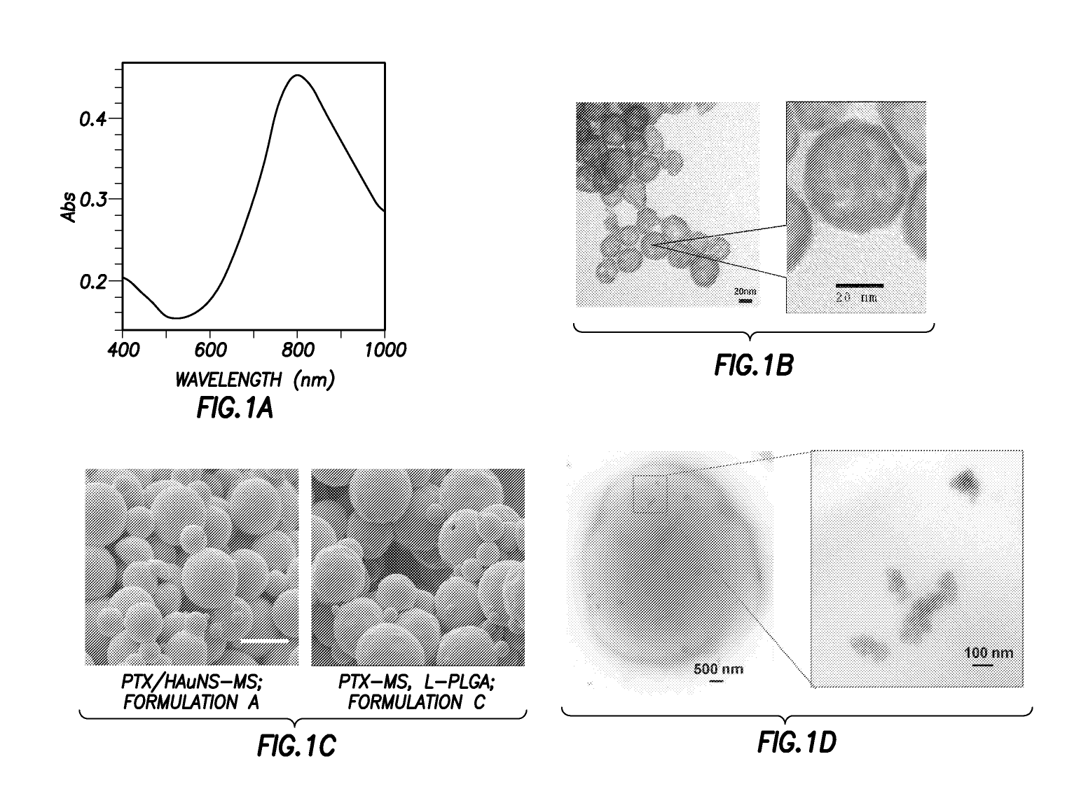

HAuNS were prepared by the cobalt nanoparticle-mediated reduction of chloroauric acid. The absorption spectrum of HAuNS showed the plasma resonance peak tuned to the NIR region (λₘₐₓ=808 nm) (FIG. 1A). TEM images revealed the near-spherical morphology of HAuNS (FIG. 1B). The average diameter of HAuNS and the thickness of the Au shell were 36 nm and 4 nm, respectively, as measured from the TEM images. This is consistent with the average diameter determined by dynamic light scattering, which yielded a mean diameter of 34 nm. However, the range of suitable diameter of HAuNS described herein can be between about 20 nm to about 80 nm, and the thickness of the Au shell may be from about 2 nm to about 8 nm.

No apparent aggregation or change in the UV-visible spectrum was observed when HAuNS were suspended in pure water at room temperature over a period of 1 month. Table 1 summarizes the parameters used in the preparation of the various microspheres.

TABLE 1
Preparation Parameters For PLGA Microspheres

| | | HAuNS loading | |
|---|---|---|---|
| | | (particles/mg | EE of PTX |
| Formulation | Microspheres | microspheres) | loading (%) |
| A | PTX/HAuNS-MS | 9.4 × 10⁹ | 102 |
| B | PTX/HAuNS-MS | 4.7 × 10¹⁰ | 97.8 |
| C | PTX-MS | — | 104 |
| D | HAuNS-MS | 9.4 × 10⁹ | |

The concentration of PLGA in the organic phase was 30% (w/v). The concentration of polyvinyl alcohol in the outer aqueous phase was 2% (w/v). The theoretical PTX loading was 5% (w/w) for all formulations. HAuNS, hollow gold nanospheres; EE, encapsulation efficiency; PTX, paclitaxel; MS, microspheres.

PTX was readily loaded into PLGA microspheres, with encapsulation efficiency (EE) close to 100% due to its hydrophobic nature. More than 90% of HAuNS were encapsulated into the microspheres by the double emulsion method (Table 1). FIGS. 1C and 1D show SEM and TEM photographs of PTX/HAuNS microspheres. The size of the microspheres was between 1-10 μm. All formulations of microspheres had a dense texture and nonporous surface. The introduction of HAuNS into PTX-loaded microspheres formed from PLGA resulted in a smoother surface than those without HAuNS (compare formulations A and C in FIG. 1C). This phenomenon is analogous to the role reinforcing steel bars play in concrete, as HAuNS nanoparticles made the PLGA microspheres denser and harder. You, et al., 288, 315-323, Int. J. Pharm. (2005). TEM photographs of PTX/HAuNS-MS revealed the dispersion of clusters of HAuNS in the PLGA polymeric matrix (FIG. 1D).

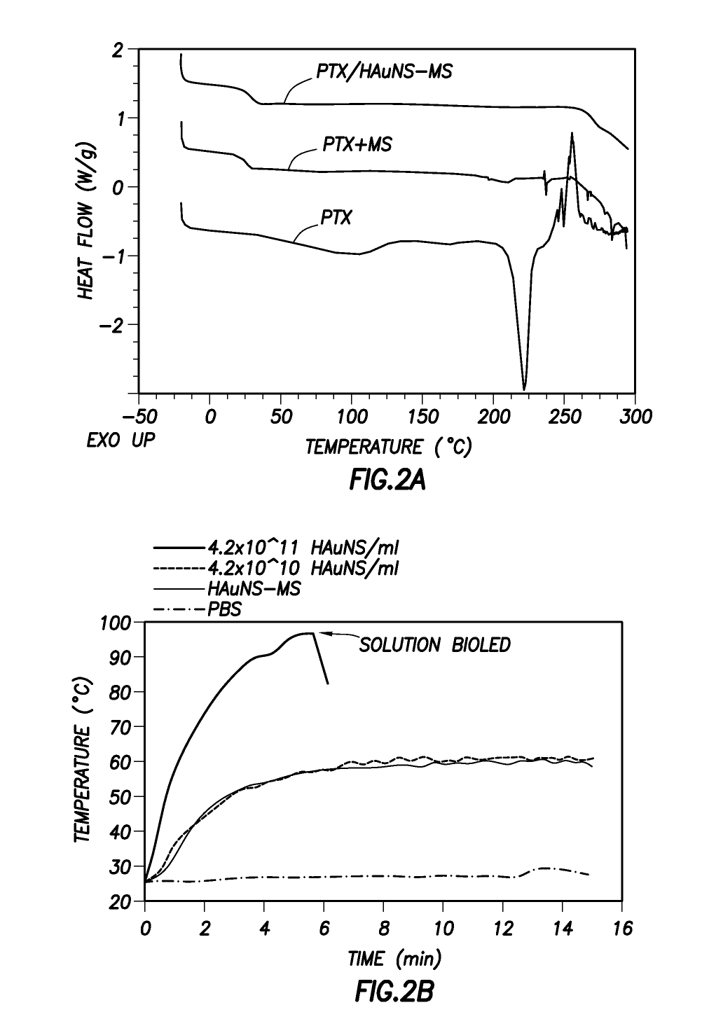

DSC thermograms for both PTX alone and the mixture of PTX and plain PLGA microspheres exhibited a single melting endothermal peak at around 210° C., just before degradation. In addition, the PTX thermogram showed an endothermic dehydration peak at ˜85° C. (FIG. 2A). These data are consistent with data in the literature for hydrated crystalline PTX. See e.g., R. T. Liggins, et al., 86, 1458-1463, J. Pharm. Sci. (1997). Both endothermic dehydration and melting peaks were missing in PTX/HAuNS-MS, suggesting that PTX was molecularly dispersed in the PLGA polymeric matrix.

Continuous exposure of aqueous suspensions of HAuNS and HAuNS-MS to NIR light resulted in the rapid elevation of their temperatures. At a laser output power of 1.5 W (˜4.5 W/cm²) and an equivalent HAuNS concentration of 4.2×10¹⁰ particles/mL, the temperatures of both suspensions (HAuNS and HAuNS-MS) were elevated 23.3° C. after 5 min of exposure. At a higher HAuNS concentration of 4.2×10¹¹ particles/mL, the aqueous solution reached the boiling point in less than 5 min. In comparison, no significant temperature change was observed when PBS was exposed to the laser light (FIG. 2B). Plain PLGA-MS in PBS at a concentration of 50 mg/mL also did not cause temperature change upon NIR light irradiation (data not shown). These results indicate that HAuNS-loaded PLGA-MS was able to elevate the temperature of water to the same extent as HAuNS alone at a concentration of 4.2×10¹⁰ nanoparticles/mL and that encapsulation of HAuNS into PLGA-MS did not adversely affect the absorption of NIR light and the photothermal conversion capacity of HAuNS.

NIR Light Triggered Release of PTX from PTX/HAuNS-MS

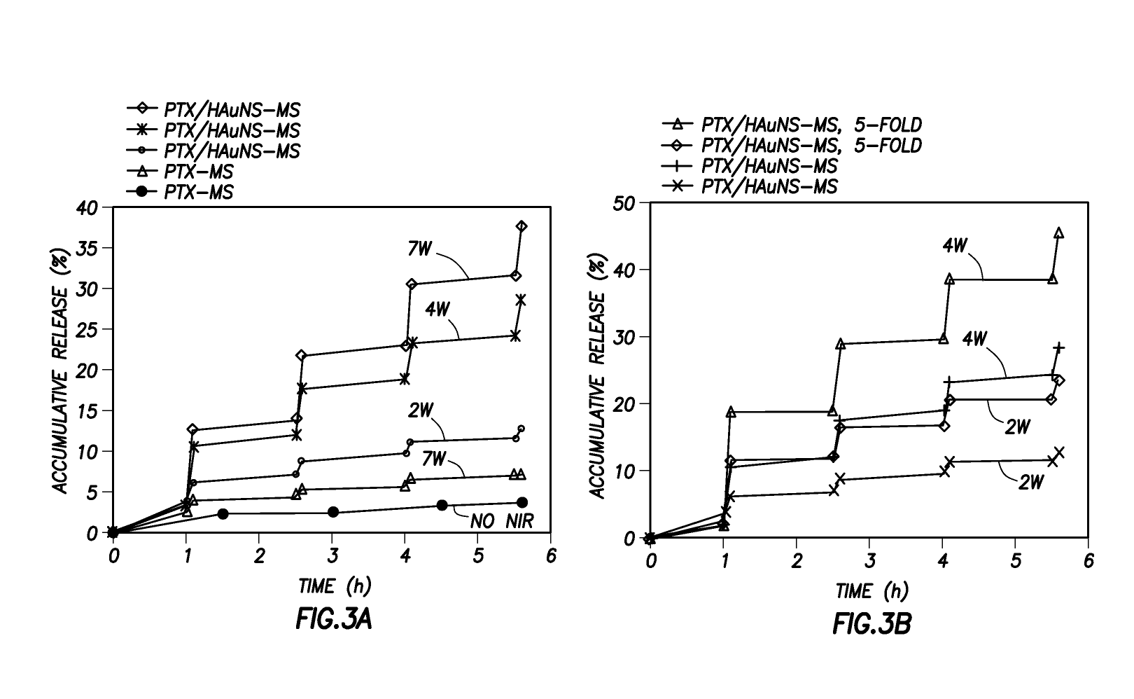

FIG. 3 shows the characteristics of PTX release from PTX/HAuNS-MS (formulations A and B) and from PTX-MS (formulation C) prepared from PLGA. The microsphere suspensions (10 mg/mL) were irradiated repeatedly using an NIR laser over a period of 5 min, followed by 1.5-h intervals with the laser turned off. FIG. 3 indicates the time periods during which the microspheres were irradiated using the NIR laser. A rapid increase in PTX release from PTX/HAuNS-MS was observed upon NIR irradiation, and the PTX release slowed significantly when NIR irradiation was switched off. After the first NIR exposure for 5 min at 7-W output power, the cumulative release, defined as the percentage of released PTX to total entrapped PTX, increased from 3.2% to 12.6%.

However, only about 1% of PTX (from 12.6% to 13.9%) was released during the subsequent 1.5 h of incubation without NIR light exposure. During the second 5-min NIR exposure, the PTX release rate was elevated rapidly, with an 8% (from 13.9% to 21.9%) increase in cumulative release. There were 7.4% and 6.2% increases in cumulative PTX release for the third and fourth NIR exposure cycles, respectively. In contrast, less than 1% of PTX was released during the 1.5-h periods without NIR light exposure in each of the following three incubation periods. The accumulated release of PTX over the whole course of the experiment approached 40% when PTX/HAuNS-MS were exposed to the NIR laser 4 times for 5 min each. In comparison, PTX-MS (without HAuNS) subjected to the same treatment protocol displayed a cumulative release of <7% over the entire experimental period. PTX-MS without laser irradiation had an accumulated release of only 3.6% (FIG. 3A).

The drug release increased with increasing NIR laser power. During the first NIR light exposure, 9.4%, 7.5%, and 2.1% of PTX was released from the PTX/HAuNS-MS at output powers of 7 W, 4 W, and 2 W, respectively (FIG. 3A). The amount of PTX released also increased with increasing HAuNS loading in the microspheres. After NIR laser exposure, more PTX was released from the PTX/HAuNS-MS containing a higher concentration of HAuNS than from those containing a lower concentration of HAuNS. For example, 17.2% of PTX was released from PTX/HAuNS-MS (formulation B) during the first NIR light irradiation at an output power of 4 W. In comparison, 9.4% of PTX was released from PTX/HAuNS-MS containing 5-fold less HAuNS (formulation A) (FIG. 3B).

### In Vitro Cytotoxicity

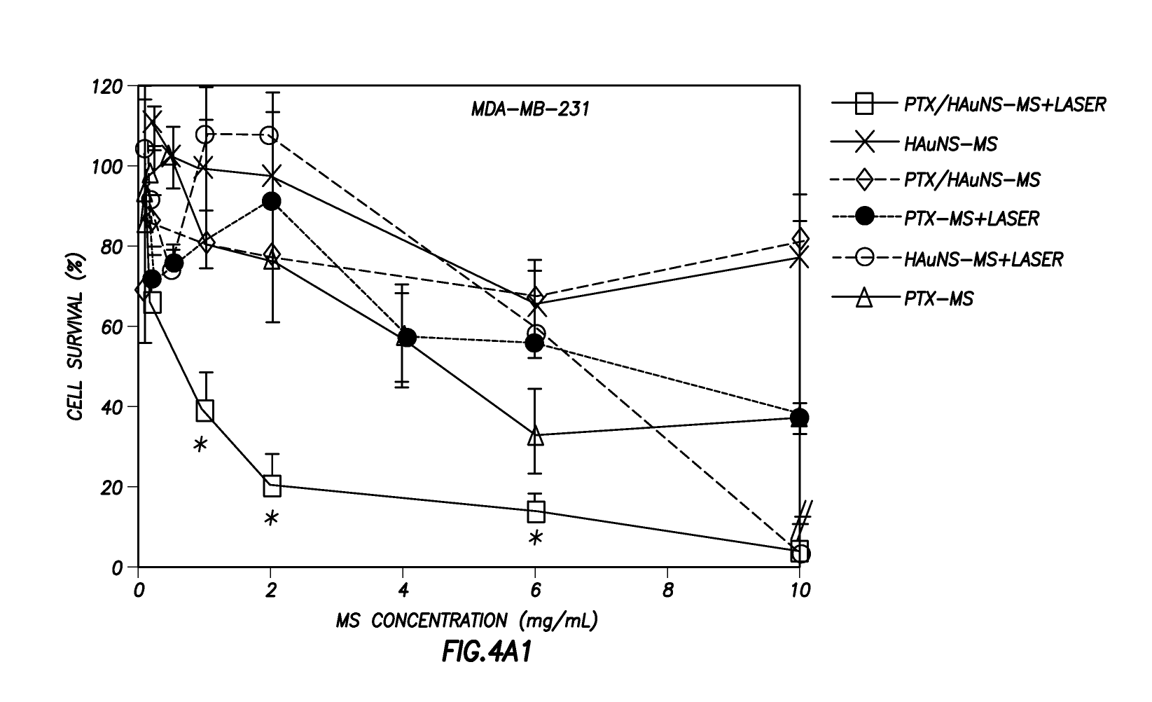

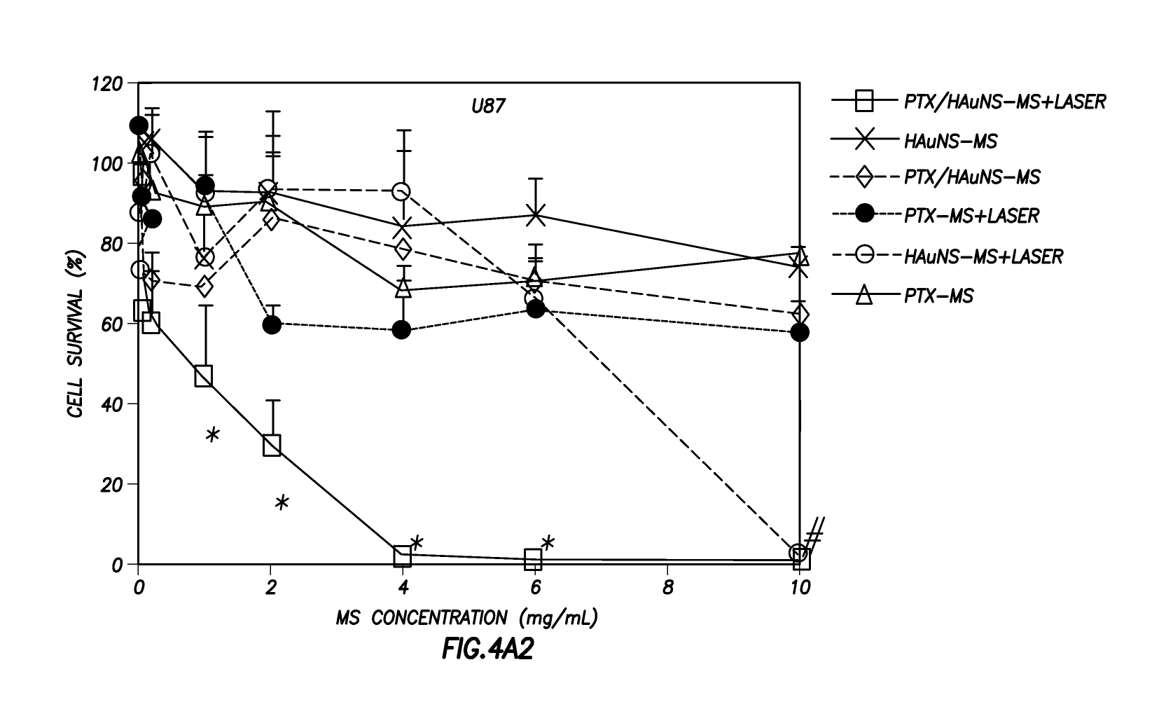

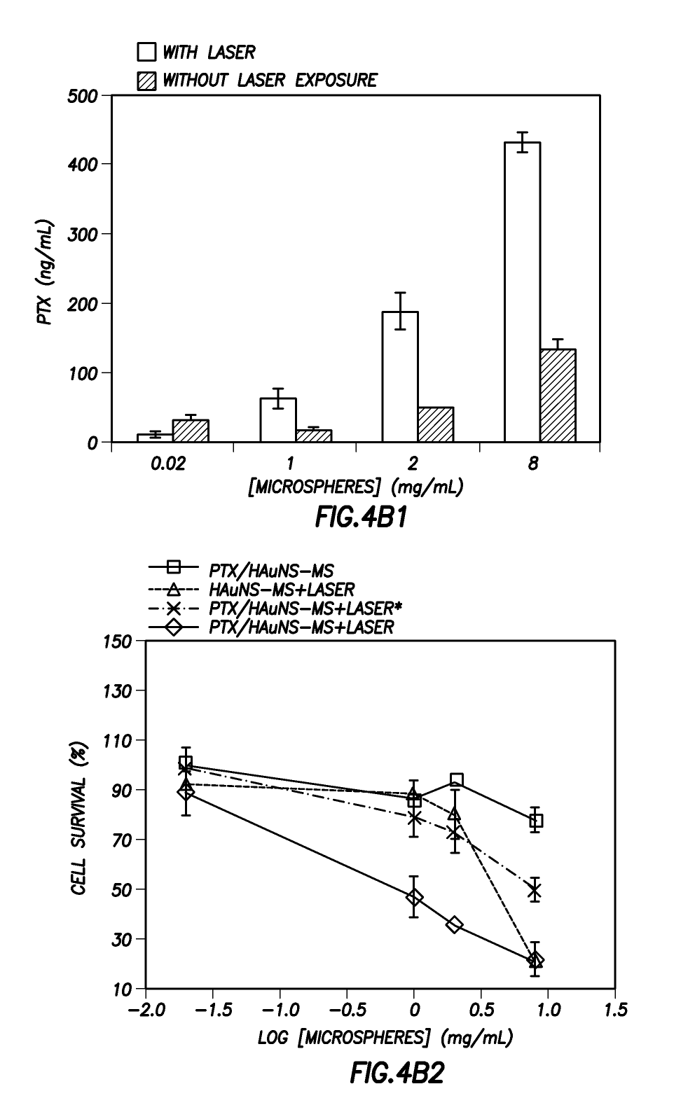

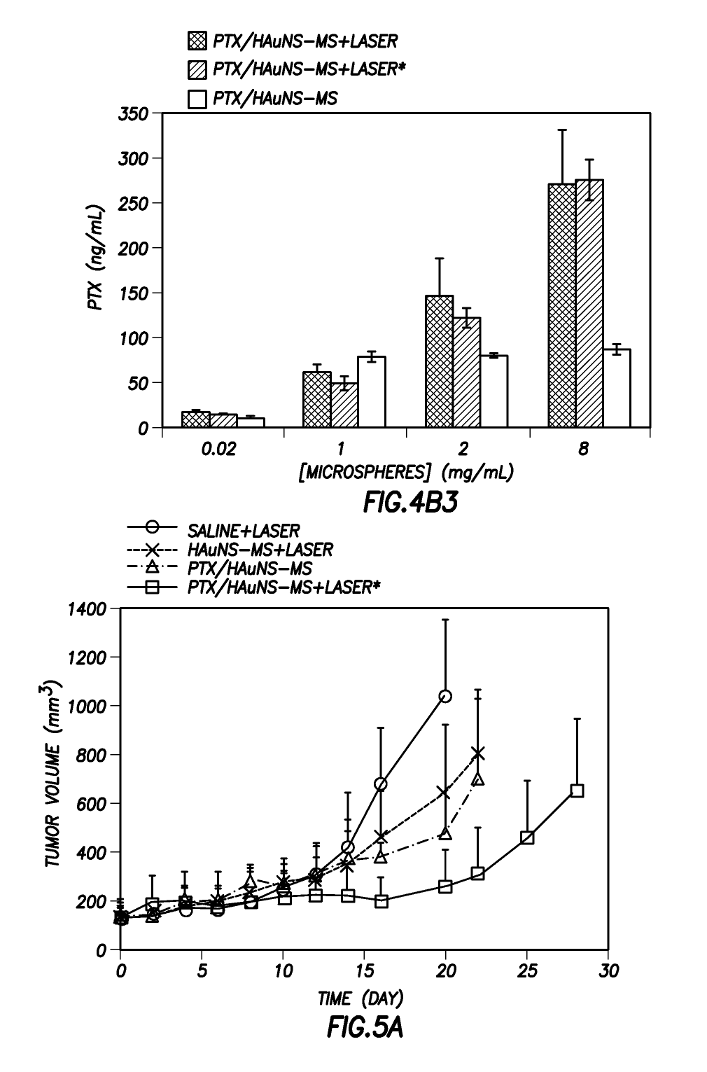

The cytotoxic effects of PTX/HAuNS-MS, with or without NIR irradiation, in MDA-MB-231 and U87 cells are shown in FIG. 4. There was a significant difference between the cell-killing effect of PTX/HAuNS-MS combined with NIR irradiation and that of PTX/HAuNS-MS alone. For example, when MDA-MB-231 cells were incubated with PTX/HAuNS-MS at concentrations of 1 mg/mL and 10 mg/mL, 80.6% and 81.6% of the cells, respectively, were viable after 72 h of incubation. However, when cells were incubated with PTX/HAuNS-MS at the same microsphere concentrations and irradiated with NIR, only 38.8% and 3.9% of the cells, respectively, survived (FIG. 4A). Moreover, when cells were incubated with HAuNS-MS (no PTX) at microsphere concentrations of 0.05-6.0 mg/mL, more than 60% of the cells were viable, regardless of whether the cells were irradiated with the NIR laser. Significant cell killing was observed with HAuNS-MS only at the highest microsphere concentration tested (10 mg/mL). Similar findings were observed with U87 cells (FIG. 4A).

To further study whether there is synergistic interaction between photothermal effect and PTX's cytotoxic effect, we conducted cytotoxicity study against MDA-MD-231 cells using culture medium pre-incubated with PTX/HAuNS-MS and treated with NIR laser. As shown in FIG. 4B, irradiation of PTX/HAuNS-MS with NIR laser at 2 W for 5 min caused significant increase of the concentration of free PTX in culture medium when the concentration of microspheres was greater than 1.0 mg/mL (FIG. 4B, top left). Treatment with PTX/HAuNS-MS alone at 0.02, 1.0, 2.0, and 8.0 mg/mL caused 0.03±2.6%, 14.3±1.3%, 6.6±1.5%, 22.2±4.8% cell killing, respectively. In comparison, when PTX/HAuNS-MS in culture medium was pre-irradiated with NIR laser, the percent of cell killed increased to 2.3±9.1%, 21.2±7.8%, 27.4±8.1%, 50.2±4.7%, respectively. The significant enhancement in cell killing did not involve photothermal killing effect, because PTX/HAuNS-MS was irradiated in the absence of cells. However, when PTX/HAuNS-MS were irradiated with NIR laser in the presence of MDA-MB-231 cells, the percent of cells killed was 10.6±9.4%, 53.2±8.2%, 64.3±0.9%, 78.4±6.9% at PTX/HAuNS-MS concentrations of 0.02, 1.0, 2.0, and 8.0 mg/mL, respectively. When cells were treated with HAuNS-MS (without PTX) and NIR laser under the same conditions, the percentages of cell killed were 7.4±5.3%, 11.4±4.8%, 19.8±9.7%, and 78.4±1.0% at HAuNS-MS concentrations of 0.02, 1.0, 2.0, and 8.0 mg/mL, respectively (FIG. 4B, top right). There was no significant difference in the concentration of free PTX in culture media after 3 days of incubation with MDA-MB-231 cells between those pre-treated with PTX/HAuNS-MS and NIR laser, and those irradiated together with PTX/HAuNS-MS and the cells (FIG. 4B, bottom).

2.4.

### In Vivo Antitumor Activity

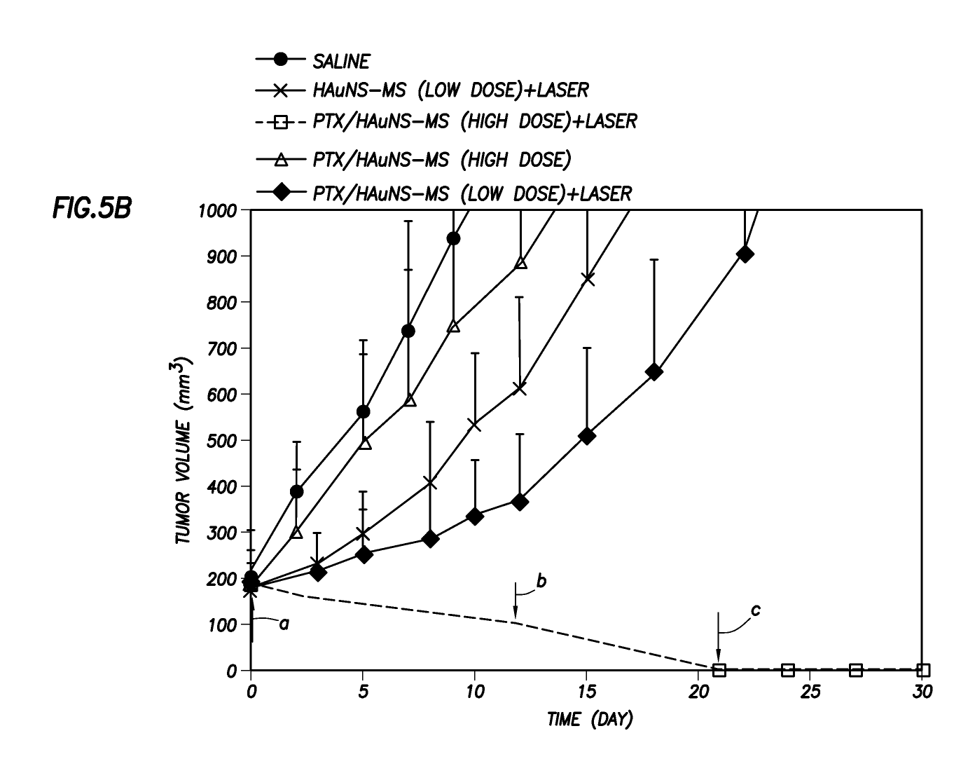

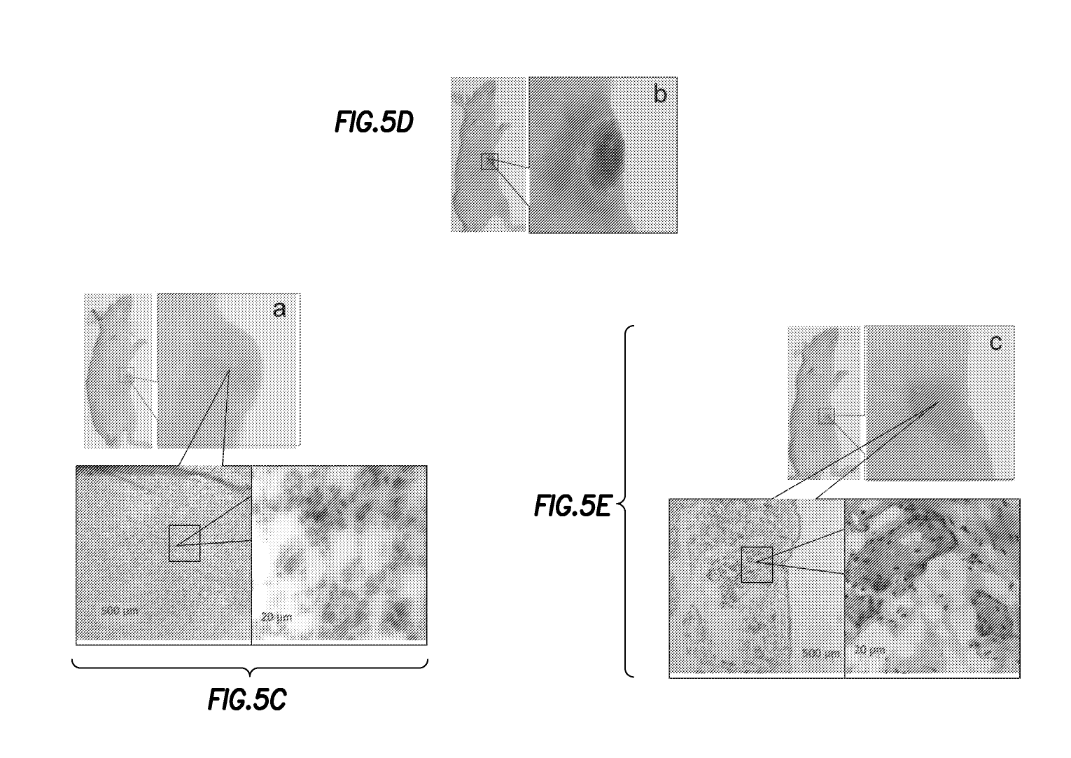

FIG. 5A shows the U87 tumor growth curves after intratumoral injection of various microspheres. The tumor growth delays, expressed as the number of days for tumors to grow from 100 mm³ to 500 mm³, were 27.5±3.8 (n=4), 21.5±1.7 (n=4), 19.0±3.5 (n=4), and 14.8±1.8 (n=5) days for mice treated with PTX/HAuNS-MS plus NIR light irradiation, PTX/HAuNS-MS, HAuNS-MS plus NIR light irradiation, and saline plus NIR light irradiation, respectively. Treatment with both PTX/HAuNS-MS and NIR laser caused a significant growth delay compared to treatments with HAuNS-MS (without PTX) plus laser (p=0.045), PTX/HAuNS-MS without laser (p=0.016), and saline plus laser (p=0.003).

The average MDA-MB-231 tumor growth curves after treatment with the microspheres at various doses are presented in FIG. 5B. The tumor growth delays were 25.0±3.5, 19.8±4.0, 15.8±6.1, and 10.2±2.8 days for mice treated with PTX/HAuNS-MS plus laser (low dose), HAuNS-MS plus NIR laser (low dose), PTX/HAuNS-MS (high dose), and saline, respectively. Similar to the findings with U87 tumors, treatment with combined PTX/HAuNS-MS and laser at a lower dose of microspheres (1.0 mg equivalent PTX/kg, 4.7×10⁹ HAuNS particles/mouse) caused significant growth delay compared to treatments with saline (p<0.0001) or HAuNS-MS plus NIR laser (p=0.037). At a higher dose of PTX/HAuNS-MS (6.0 mg equivalent PTX/kg, 2.82×10¹⁰ HAuNS particles/mouse), NIR laser irradiation caused burning of tissue and extensive necrosis in the first 3 weeks after the initiation of treatment. The burned tissues gradually healed, however, and by 20 days after treatment, all tumors had disappeared completely, with only scar tissue remaining. Histologic examination confirmed the absence of tumor cells in the remaining scar tissue (FIG. 5C). In contrast, mice treated with the same dose of PTX/HAuNS-MS, but without NIR laser irradiation, showed minimal antitumor activity; the tumor growth delay, measured in days for tumors to grow from 200 mm³ to 1000 mm³, did not differ significantly from that in mice treated with saline control (p=0.056) (FIG. 5B).

Despite advances over the past few decades, spatial and temporal control of drug release characteristics remains a challenge. HAuNS are nanoparticles which possess excellent colloidal stability, are small in size (typically 35-40 nm in diameter), and display tunable and strong absorption bands in the NIR region. For example, HAuNS coated with a monoclonal antibody or peptides can be useful to targeted to solid tumors after systemic administration for enhanced photothermal ablation therapy. W. Lu, et al., 15, 876-886, Clin. Cancer Res. (2009); M. P. Melancon, et al., 7, 1730-1739, Mol. Cancer. Ther. (2008). However, by incorporating HAuNS into microspheres (such as PLGA microspheres), the smaller size and strong surface plasma absorption of HAuNS results in a drug delivery system that is highly responsive to NIR laser irradiation.

Different types of drugs may be used in the combined HAuNS-MS drug delivery system described herein. Both hydrophobic drugs and soluble drugs may be used. In addition, drugs having both positive charge and negative charge can be used.

Paxlitaxel (“PTX”) is exemplary of a hydrophobic drug that can be incorporated into the HAuNS-containing PLGA microspheres and is demonstrative of the NIR light-triggered release of a highly hydrophobic drug. PTX is a widely used anticancer agent with demonstrated antitumor efficacy against breast, ovarian, lung, and head and neck cancers. E. K. Rowinsky, et al., 82, 1247-1259, J. Natl. Cancer Inst. (1990); Q. Chu, et al., 50, 355-374, Lung Cancer. (2005); D. Schrijvers, et al., 17, 218-224, Curr. Opin. Oncol. (2005); E. Saloustros, et al., 9, 2603-2016, Exp. Opin. Pharmacother. (2008).

The size of the microspheres useful in the drug delivery system presented herein depends upon how the microspheres are applied. If the microspheres are intended for intracellular delivery of anticancer agents (i.e. paclitaxel), drug/HAuNS-embedded particles of smaller size (<1 μm) may result in more efficient cellular uptake. If the intended application is for intratumoral delivery, release of the anticancer drugs in the interstitial space with microspheres of larger sizes (<1 μm) may be sufficient. The cellular uptake and antitumor efficacy of drug/HAuNS-embedded particles of different sizes can vary different applications. The detailed mechanism of drug release, particularly the effect of HAuNS-mediated photothermal response on the mobility of polymer chains and permeability to hydrophobic drugs depends on the particular application.

As noted above, FIG. 6 depicts the proposed mechanism of NIR light-triggered release of drugs from PLGA microspheres, as well as the hypothetical structure of microspheres containing HAuNS and PTX. Because the microspheres were prepared by a W1/O/W2 double-emulsion solvent evaporation method, it was anticipated that PTX dissolved in the organic phase together with the PLGA polymer would be uniformly distributed within the polymer, whereas HAuNS nanoparticles dissolved in the original aqueous phase would be dispersed in the water phase inside the microspheres (FIG. 6).

The structural features of PTX/HAuNS microspheres depicted in FIG. 6 are supported by TEM study, which showed the formation of HAuNS clusters within the microspheres, possibly as a result of the inner water droplets containing a high local concentration of HAuNS (FIG. 1D), and by DSC analysis, which revealed that the liposoluble PTX was dispersed in PLGA polymer at the molecular level (FIG. 2A). HAuNS recovered from PTX/HAuNS microspheres had the same absorption property as HAuNS shown in FIG. 1A (data not shown). Thus the encapsulation process did not affect the optical property of HAuNS. In vitro studies confirmed that HAuNS-containing microspheres were capable of raising the temperature of the aqueous solution as much as did plain HAuNS at the same concentration (FIG. 2B). Importantly, the heat generated was sufficient for triggering PTX release. The release of PTX from microspheres containing HAuNS could be readily modulated by controlling the NIR laser output power, the duration and frequency of laser irradiation, and the HAuNS payload in the microspheres (FIG. 3). These results indicate that PTX release from PTX/HAuNS-MS resulted directly from the photothermal effect mediated by HAuNS embedded in the microspheres. The polymer chains became increasingly more flexible due to a rapid elevation of the local temperature of the microspheres upon NIR irradiation, resulting in increased release of the PTX drug molecules from the PLGA polymeric matrix (FIG. 6).

In vitro cytotoxicity studies confirmed that the antitumor effect of PTX/HAuNS-MS was enhanced when cells were irradiated with NIR laser. Significant cell killing was observed with PTX/HAuNS-MS plus NIR irradiation, but not with PTX/HAuNS-MS alone or PTX-MS alone, indicating that the amount of PTX released from the PTX-containing microspheres in the absence of NIR light was insufficient to kill tumor cells. In addition, PTX/HAuNS-MS plus NIR irradiation, but not HAuNS-MS plus NIR laser, caused significant cytotoxicity at microsphere concentrations of up to 6 mg/mL (0.3 mg equivalent PTX/mL), indicating that PTX released from PTX/HAuNS-MS was primarily responsible for the cell killing achieved with PTX/HAuNS-MS plus NIR at these concentrations. At higher concentrations of HAuNS-MS (≧10 mg/mL), the photothermal effect mediated by HAuNS was sufficient to ablate the tumor cells (FIG. 4A). Further studies suggest that at microspheres concentrations of 1.0 and 2.0 mg/mL, treatments with PTX/HAuNS-MS plus NIR laser caused greater cell killing effect than combined cell killing effect achieved with HAuNS-MS plus laser (pure photothermal killing effect) and with culture media containing PTX/HAuNS-MS pre-exposed to NIR laser (pure cytotoxic effect of the drug) (FIG. 4B). Because there was no difference in drug concentration after 72 h cell incubation between the culture medium that was irradiated with PTX/HAuNS-MS in the absence of cells and the culture medium that was irradiated with PTX/HAuNS-MS in the presence of cells, the observed enhanced cell killing with PTX/HAuNS-MS plus laser could be attributed to a synergistic interaction between PTX and photothermal effect mediated through PTX/HAuNS-MS.

Enhanced antitumor activity with PTX/HAuNS-MS followed by NIR irradiation was also demonstrated in vivo in experimental tumor models. Significantly higher antitumor activity against subcutaneously inoculated U87 tumors was achieved with combined PTX/HAuNS-MS and NIR laser than with PTX/HAuNS-MS alone (low PTX release rate and no photothermal effect) or with HAuNS-MS plus NIR laser (photothermal effect but without PTX) (FIG. 5A). Similar findings were observed in an orthotopically inoculated mammary MDA-MB-231 tumor model (FIG. 5B). Notably, at a high dose of PTX/HAuNS-MS (6.0 mg equivalent PTX/kg), cure of tumors was achieved with PTX/HAuNS-MS combined with NIR laser, whereas PTX/HAuNS-MS alone at the same dose had little antitumor effect. The enhanced antitumor activity in tumors treated with combined PTX/HAuNS-MS and NIR is most likely a result of both the cytotoxic effect of PTX release from the microspheres induced by NIR light and the photothermal effect mediated by HAuNS embedded in the microspheres.

Novel polymeric drug delivery systems, including injectable microspheres, have been developed for local delivery of PTX in order to meet the challenges encountered in the clinical use of PTX, i.e., hypersensitivity reactions and cumulative toxicity. J. K. Jackson, T. Hung, K. Letchford, H. M. Burt, Int. J. Pharm. 2007, 342, 6-17. Owing to the strong hydrophobic interaction between PTX and hydrophobic PLGA polymer, PTX release from this commonly used biodegradable polyester has proven to be extremely slow. To enhance the release of PTX from PLGA microspheres, PLGA was blended with low-molecular-weight amphipathic diblock copolymers. This resulted in an up to 20-fold increase in the burst effect. Hence, the release of PTX from PLGA microspheres is uncontrollable and unpredictable, and only a small portion of the drug is eventually released, limiting the therapeutic efficacy of such methods. In this study, we demonstrated not only that NIR light could modulate release of PTX from PLGA microspheres in a controlled fashion but also that the approach significantly enhanced the antitumor efficacy of PTX-PLGA microspheres in vivo. The feasibility of enhancing the antitumor effect with light-modulated drug release in vivo is a novel, viable one. Furthermore, there is a therapeutic advantage to combining photothermal ablation therapy with NIR light-modulated drug release.

Sanchez-Iglesias et al. recently describes a novel colloidal composites containing gold nanoparticle cores covered by a thin layer of metallic nickel and a poly(N-isopropylacrylamide) (pNIPAM) shell. These authors showed that the pNIPAM shell could be swollen or collapsed as a function of temperature, thus allowing capture and release of various types of molecules. A. Sanchez-Iglesias, et al., 3, 3184-3190, ACS Nano (2009). Conversely, by incorporating hollow gold nanospheres into biodegradable PLGA microspheres, photothermal effect could be modulated with NIR light, which is the optimal wavelength that allows penetration of light deep into soft tissues (˜5 cm).

Hence, the drug delivery system and methods of modulating drug release using NIR light as the external stimuli presented herein can provide for rapid and repetitive drug release, readily achieved upon NIR irradiation. Owing to the ability of NIR to penetrate deep tissues, modulation of drug release with NIR light can find applications in the treatment of cancer and other diseases. In addition, specifically, intratumoral administration of the microspheres for anticancer therapy proves that the microspheres containing HAuNS and anticancer agents may be useful in chemo-embolization applications where controllable and repetitive release of anticancer drugs in response to an NIR laser beam is needed.

### Example II

### Materials and Experiments

### Reagents

DOX, methoxy-PEG-SH (MW, 5,000), PBS (pH 7.4), and the cell cytotoxicity kit for MTT assay were obtained from Sigma-Aldrich (St. Louis, Mo.). Trisodium citrate dehydrate (>99%), cobalt chloride hexahydrate (99.99%), sodium borohydride (99%), and chloroauric acid trihydrate (ACS reagent grade) were purchased from Fisher Scientific (Pittsburgh, Pa.) and were used as received. Methylene chloride (ACS grade) was obtained from Baxter Healthcare Corp (Deerfield, Ill.). Gold nanoparticles with average diameter of 41.5 nm were purchased from BB International (Madison, Wis.).

### Cell Culture

MDA-MB-231 (human breast carcinoma) cells were maintained at 37° C. in a humidified atmosphere containing 5% CO₂ in Dulbecco's modified Eagle's medium and 10% fetal bovine serum (Life Technologies, Inc., Grand island, NY).

### Synthesis of HAuNS and Conjugation of PEG to HAuNS

HAuNS were synthesized according to a previously reported method. Lu, W., et al., Targeted Photothermal Ablation of Murine Melanomas with Melanocyte-Stimulating Hormone Analog-Conjugated Hollow Gold Nanospheres, 15 (3), 876-86 (Clin Cancer Res 2009). Briefly, cobalt nanoparticles were first synthesized by deoxygenating deionized water containing 4.5 mL of 1 mol/L sodium borohydride, 2.8 mL of 0.1 mol/L sodium citrate, and 1.0 mL of 0.4 mol/L cobalt chloride. After chloroauric acid was added into the solution containing cobalt nanoparticles, the cobalt immediately reduced the gold ions onto the surface of cobalt nanoparticles, while at the same time it was oxidized to cobalt oxide. Any remaining cobalt core was further oxidized by air, resulting in the final product, HAuNS. The size of HAuNS was determined using dynamic light scattering on a Brookhaven 90 plus particle size analyzer (Holtsville, N.Y.). UV-visible spectroscopy was recorded on a Beckman Coulter DU-800 UV-visible spectrometer (Fullerton, Calif.). The morphology of HAuNS was examined using a JEM 1010 transmission electron microscope (JEOL USA, Peabody, Mass.). The concentration of HAuNS was estimated by our previously reported method. Melancon, M. P. et al., In vitro and in vivo Targeting Of Hollow Gold Nanoshells Directed At Epidermal Growth Factor Receptor For Photothermal Ablation Therapy, 7 (6), 1730-1739, Mol Cancer Ther (2008). PEG-modified HAuNS (PEG-HAuNS) were prepared according to our previous studies. Lu, W., et al., Targeted Photothermal Ablation Of Murine Melanomas With Melanocyte-Stimulating Hormone Analog-Conjugated Hollow Gold Nanospheres, 15 (3), 876-86, Clin Cancer Res (2009) Briefly, HAuNS (5.0×10¹² particles/mL) were added to argon-purged aqueous solution containing PEG-SH with various concentrations. The reaction was allowed to proceed overnight at room temperature. For purification, the reaction mixture was centrifuged at 14,000 rpm for 20 min, and the resulting pellet was resuspended with deionized water. The process was repeated twice to remove unreacted PEG molecules.

DOX Loading onto HAuNS and PEG-HAuNS

Aliquots of free DOX in water (1.0 mM, 0.02-0.3 mL) were added into an aqueous solution of HAuNS or PEG-HAuNS (1.0×10¹¹ particles, 0.1 mL), and the mixtures were stirred at room temperature for 24 h. After centrifugation (14,000 rpm for 20 min), the precipitate was washed with PBS and centrifuged, and the washing cycle was repeated until the supernatant became colorless. All supernatants collected were pooled together, and the amount of free DOX in the supernatant was determined by spectrophotometry at 287 nm. DOX loading capacity (LC) was estimated using two methods. The first method indirectly measures attached DOX by determining the amount of unbound DOX in the supernatant according to Equation 1. The second method directly quantifies DOX attached to HAuNS after extraction of DOX from dried HAuNS with dimethyl sulfoxide according to Equation 2.

LC_{indirect}=(Total DOX used−DOX in supernatant)/(Total Au used+Total DOX used−DOX in supernatant)×100% Equation 1

LC_{direct}=Total DOX extracted from HAuNS/Total particle weight×100% Equation 2

DOX Release from HAuNS and PEG-HAuNS

The release studies were performed at room temperature. DOX@HAuNS or DOX@PEG-HAuNS (˜1.0×10¹² particles/mL) was dispersed in 2.0 mL PBS (10 mM, pH 7.4) in a 5-mL test tube. A laser probe (10-mm spot diameter) was placed on the side 5.0 cm from the center of the test tube. At predetermined time intervals, the samples were irradiated with NIR laser centered at 808 nm at an output power of 2.0 W/cm² for 3 min (Diomed 15 plus, Cambridge, UK). The nanoparticle solutions were centrifuged (14,000 rpm, 20 min) and supernatants withdrawn for analysis of DOX before and after NIR laser irradiation. The concentration of DOX in the supernatant was determined spectrophotometrically.

### Cell Uptake of DOX@HAuNS and DOX@PEG-HAuNS

MDA-MB-231 cells were transferred and cultured onto 20-mm glass cover slips in a 24-well plate and allowed to grow for 2 days. Then the medium was replaced with 1 mL of fresh culture medium containing free DOX, DOX@HAuNS, or DOX@PEG-HAuNS. After incubation for 1 hr or 48 h, the cell monolayer on the cover slip was removed, repeatedly rinsed with PBS, and then mounted for microscopic examination. The cellular fluorescence and dark-field light scattering images were examined under a Zeiss Axio Observer.Z1 fluorescence microscope (Carl Zeiss MicroImaging GmbH, USA) equipped with a dark-field condenser.

In a separated experiment, MDA-MB-231 cells cultured on 20-mm glass cover slips were irradiated with NIR laser (1.0 W/cm² for 3 min per treatment, 4 treatments in 2 h). Cell nuclei were stained with DAPI. The cellular fluorescence and light scattering images were obtained as described in the preceding paragraph.

### Cytotoxicity of DOX-HAuNS and DOX-PEG-HAuNS

A total of 2.0×10⁴ cells were plated in 96-well plates and incubated for 24 h to allow the cells to attach. The cells were exposed to free DOX, DOX@HAuNS, or DOX@PEG-HAuNS with various DOX concentrations. The cells were or were not irradiated with NIR laser at an output power of 2 W/cm² (3 min per treatment, 4 treatments in 2 h). Then the cells were incubated at 37° C. for a further 48 h. Cell survival efficiency was measured using the MTT assay according to the manufacturer suggested procedures. The data reported represented the means of triplicate measurements. In a separate experiment, the cells were exposed to HAuNS with various Au concentrations and then were or were not irradiated with NIR laser under the same conditions. Cell survival efficiency was measured after the incubation at 37° C. for 48 h.

Dual-functional hollow gold nanospheres (HAuNS, ˜40-nm diameter) capable of mediating both photothermal ablation of cancer cells and drug release upon near-infrared (NIR) light irradiation were tested. As high as 63% DOX by weight (˜1.7 μg DOX/μg Au) could be loaded to polyethylene glycol (PEG)-coated HAuNS since DOX was coated to both the outer and the inner surfaces of HAuNS. Irradiation with NIR laser induced photothermal conversion, which triggered rapid DOX release from DOX-loaded HAuNS. The release of DOX was also pH-dependent, with more DOX released in aqueous solution at lower pH. Significantly greater cell killing was observed when MDA-MB-231 cells incubated with DOX-loaded HAuNS were irradiated with NIR light, attributable to both HAuNS-mediated photothermal ablation and cytotoxicity of released free DOX.

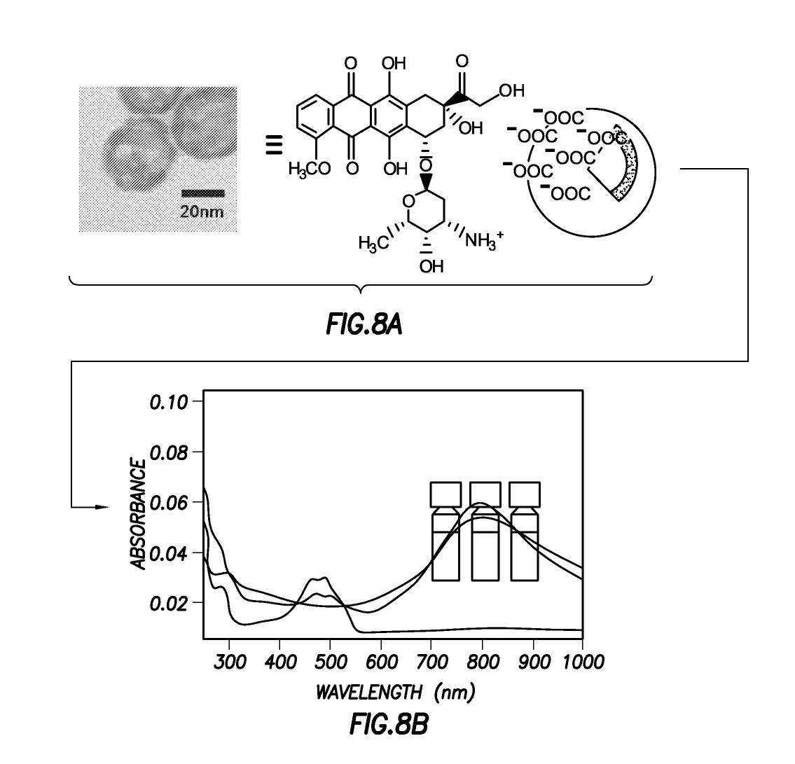

As shown in FIG. 8, hollow gold nanospheres with strong surface plasmon absorption in the near-infrared region formed stable complexes with doxorubicin with extremely high payload, and DOX was released intracellularly upon near-infrared light irradiation.

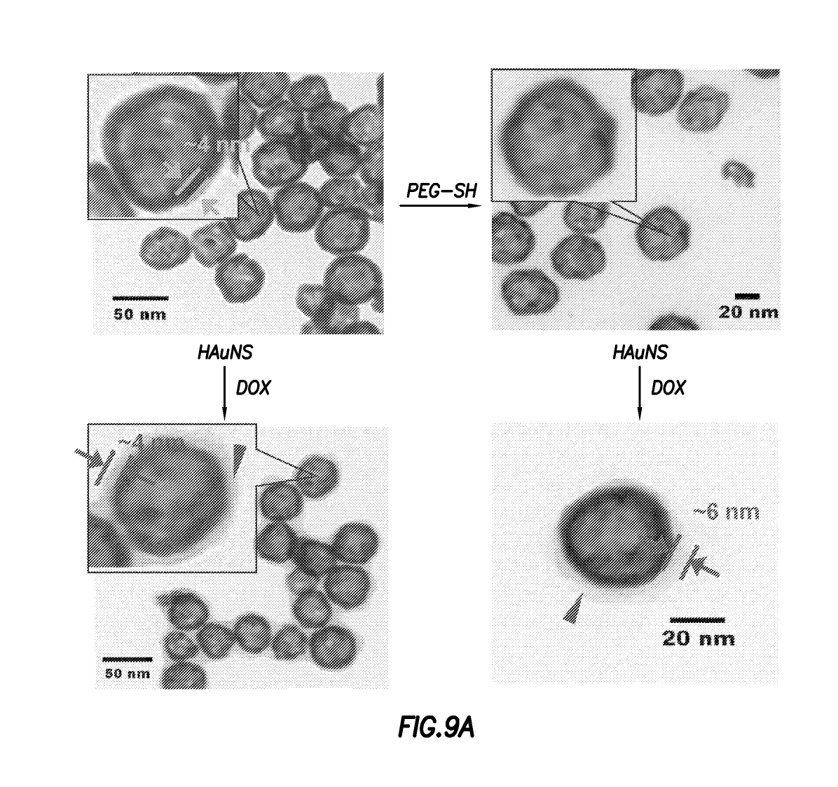

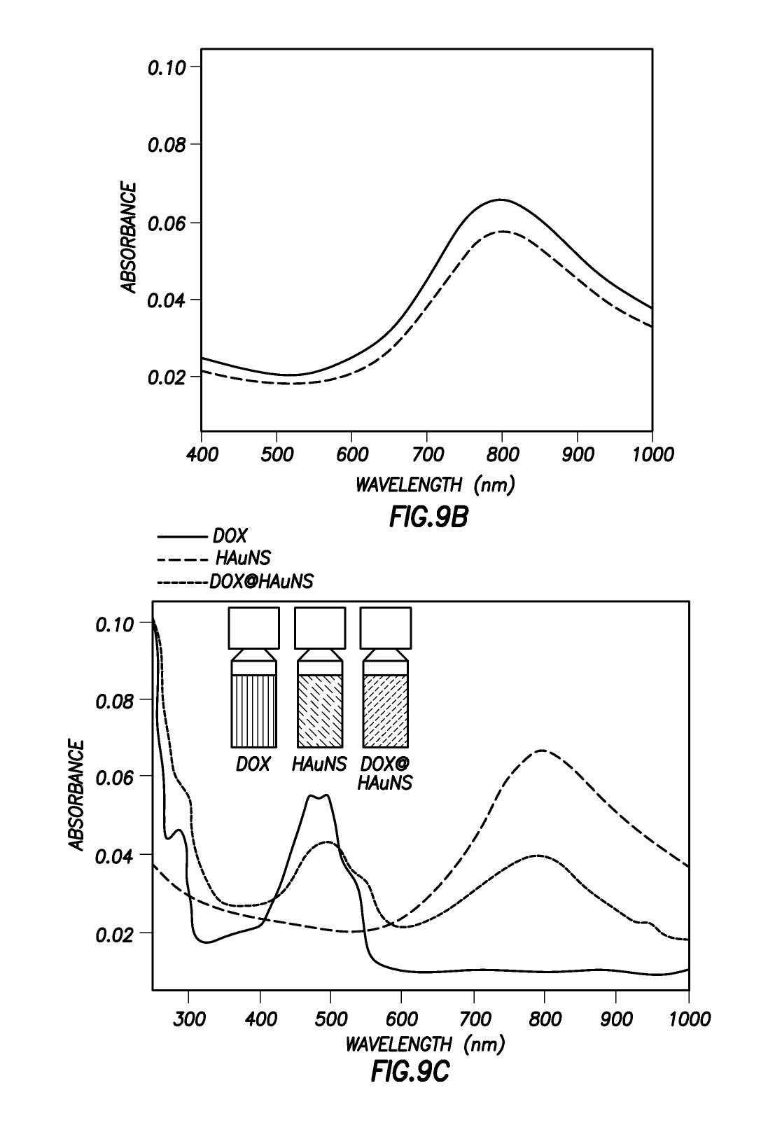

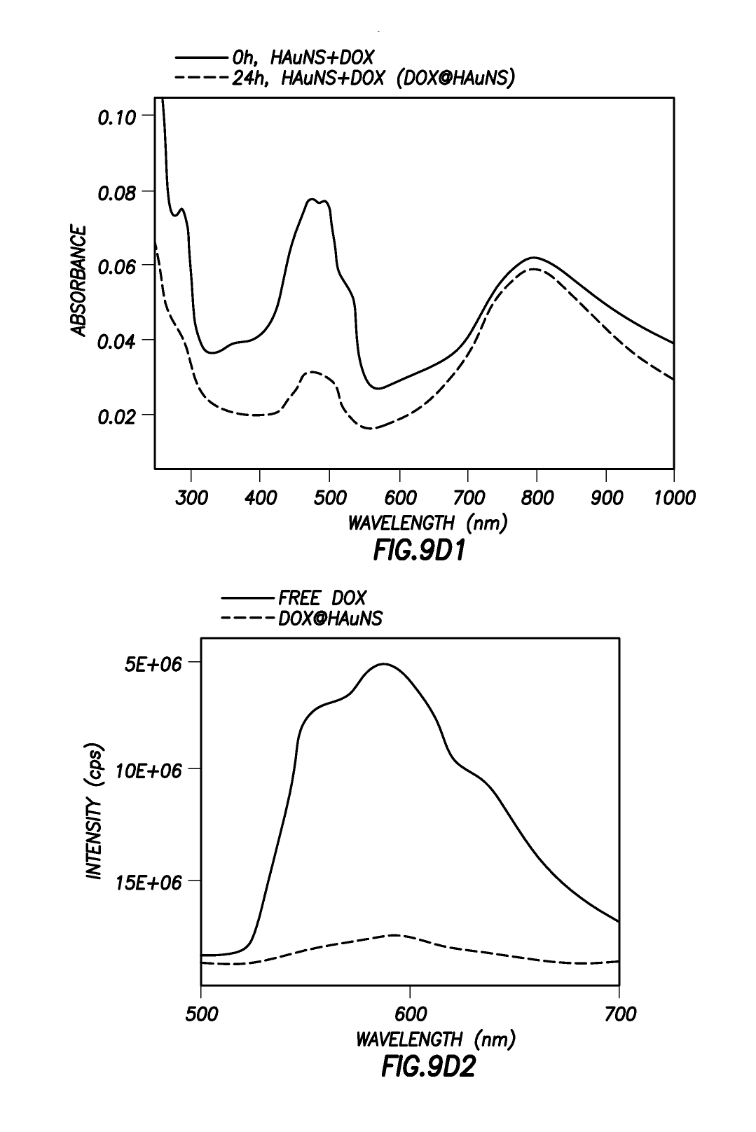

HAuNS were synthesized by sacrificial galvanic replacement of cobalt nanoparticles in the presence of chloroauric acid according to the method of Schwartzberg et al. Schwartzberg, A. M. et al., Synthesis, Characterization, And Tunable Optical Properties Of Hollow Gold Nanospheres, 110 (40), 19935-19944 (J Phys Chem 2006) The average diameter of the resulting HAuNS was 38.5±1.7 nm, as determined by the dynamic light scattering method. Transmission electron microscopy (TEM) revealed the morphology of the HAuNS (FIG. 9A) and indicated that they had average diameter of 43±4.6 nm and average Au shell thickness of 4.0 nm. Surface modification by polyethylene glycol (PEG) resulted in a moderate increase in the average diameter of the HAuNS from 38.5±1.7 nm to 48.6±1.3 nm. Compared with HAuNS, PEG-HAuNS had a significantly increased colloidal stability: no aggregation was observed when PEG-HAuNS were stored in water at room temperature over a period of 3 months. The absorption spectra showed that the plasma resonance peaks for both HAuNS and PEG-HAuNS were tuned to the NIR region (˜800 nm) (FIG. 9B). Thus, PEG modification did not affect the spectrum characteristic of HAuNS but did increase their physical stability.

The complexes of DOX@HAuNS and DOX@PEG-HAuNS were readily formed by simple mixing of HAuNS or PEG-HAuNS solutions with DOX for 24 h at room temperature and then repeated washing to remove unbound DOX. TEM images clearly showed a DOX layer with a thickness of about 4-6 nm covering the surface of both DOX@HAuNS and DOX@PEG-HAuNS, which did not exist in HAuNS and PEG-HAuNS prior to DOX coating (FIG. 9A). The color of the HAuNS changed from greenish to henna upon absorption of DOX. DOX@HAuNS displayed a UV-V is absorption peak at 490 nm characteristic of DOX and a broad NIR plasmon absorption peak at ˜800 nm characteristic of HAuNS (FIG. 9C). At 24 h after mixing, the absorbance peak intensity of DOX in the UV-visible region was significantly reduced compared with the absorbance peak intensity immediately after mixing of DOX and HAuNS (0 h), when DOX was unbound (FIG. 9D). In addition, compared to free DOX, which exhibited strong fluorescence emission, the fluorescence signal from DOX in DOX@HAuNS was almost completely quenched (FIG. 9D, inset), a phenomenon known to occur when fluorophores are attached to a metal nanoparticle surface with close proximity. Fan, C., et al., Beyond Superquenching: Hyper-Efficient Energy Transfer from Conjugated Polymers to Gold Nanoparticles, 100 (11), 6297-301, Proc Natl Acad Sci USA (2003). These results indicate that DOX was tightly bound to HAuNS and PEG-HAuNS after 24 h incubation.

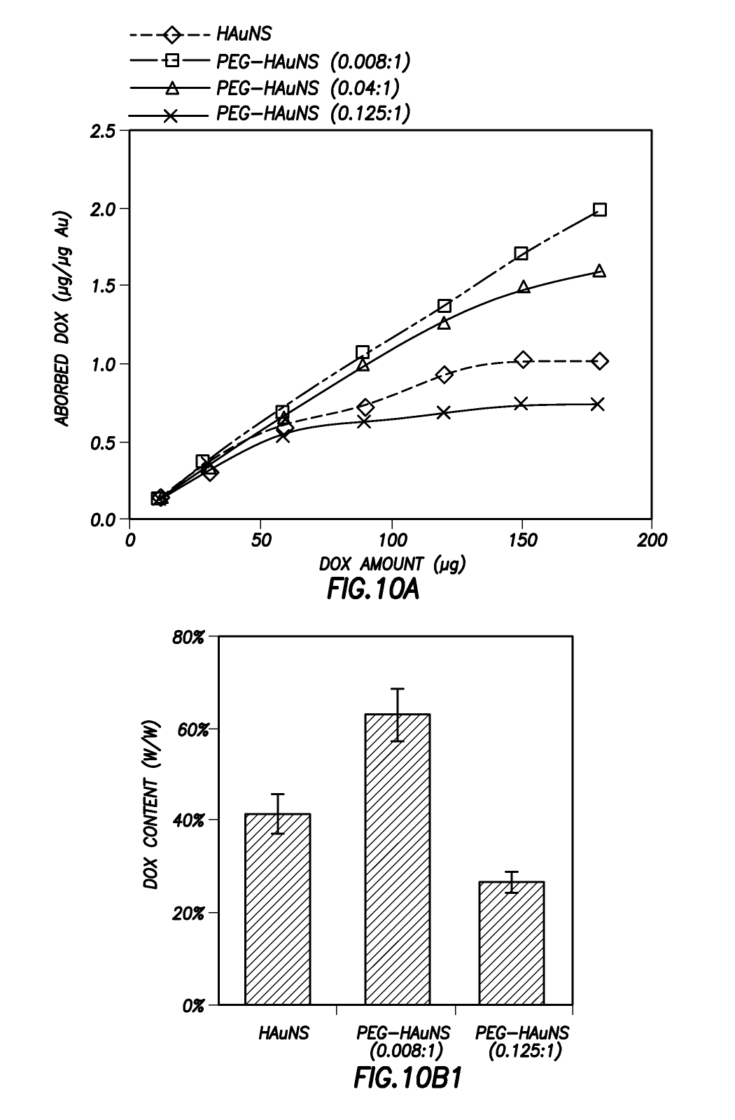

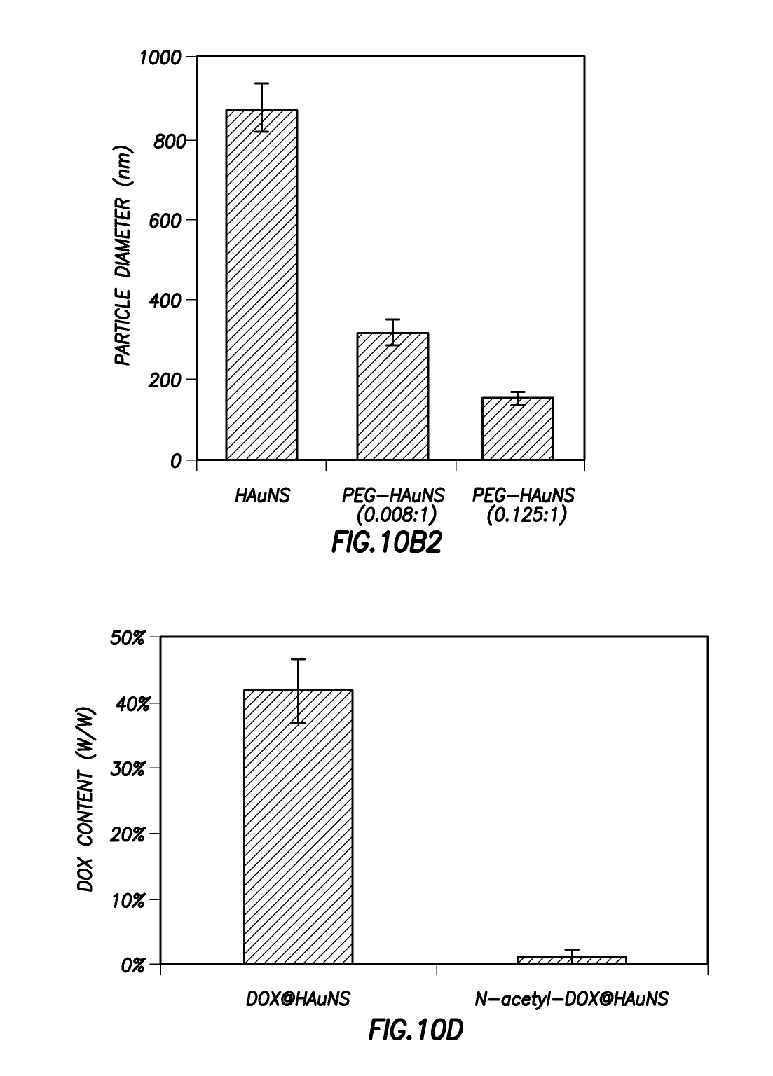

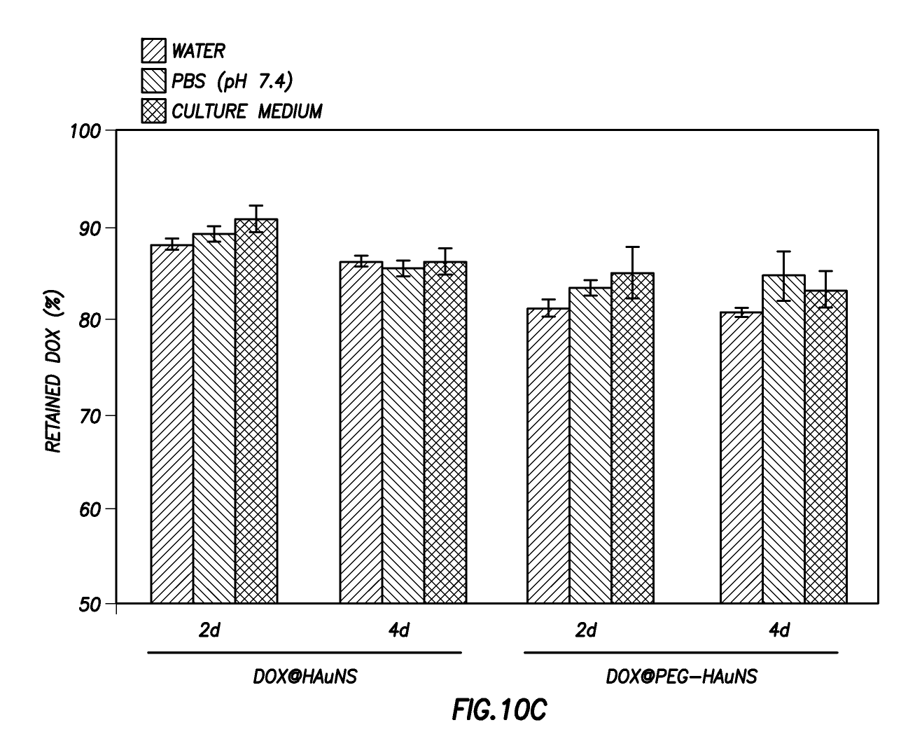

FIG. 10A shows Langmuir adsorption isotherms at room temperature for the adsorption of DOX by HAuNS and PEG-HAuNS. PEG modification on HAuNS significantly affected DOX's absorbing efficiency. At a lower PEG coating density (PEG:Au molar ratio=0.008:1), PEGylation of HAuNS resulted in higher DOX loading. However, the loading efficiency of DOX decreased with increasing PEG coating density. This may be explained by increased surface area as a result of increased colloidal stability and thus reduced population of aggregates formed among HAuNS when a small amount of PEG was introduced to the HAuNS surface. With excessive PEG modification, however, interaction between HAuNS and DOX was impeded because the steric hindrance of PEG prevented DOX molecules from approaching the surface of HAuNS. The saturation of DOX absorption to PEG-HAuNS at higher PEG coating densities (PEG:Au molar ratio=0.04:1 and 0.125:1) suggests that the surface of HAuNS was completely occupied by DOX and PEG molecules under such conditions, whereas at the lower PEG coating density (PEG:Au molar ratio=0.008:1), the DOX absorption isotherm did not plateau up to an initial DOX amount of 180 μg.

Further increase in DOX payload is possible for this particular formulation. At the DOX amount of 180 μg in the initial DOX-HAuNS mixtures, the drug contents absorbed to HAuNS were 41%, 63%, and 27% for HAuNS, PEG-HAuNS (PEG:Au molar ratio=0.008:1), and PEG-HAuNS (PEG:Au molar ratio=0.125:1), respectively, corresponding to weight ratios of 0.69, 1.7, and 0.37 between DOX and gold (FIG. 10B). DOX loading to HAuNS and PEG-HAuNS led to an increase in the size of the nanoparticles, suggesting that the presence of DOX reduced the colloidal stability of these nanoparticles. DOX@HAuNS having higher PEG density were more stable than DOX@HAuNS having lower PEG density, which in turn were more stable than HAuNS without PEG coating (FIG. 10B). In the following descriptions of our studies, “PEG-HAuNS” refers to PEG-HAuNS with PEG:Au molar ratio of 0.008:1 unless specified otherwise.

In addition to colloidal stability, we also examined the stability of absorbed DOX on HAuNS and PEG-HAuNS. After an initial release of 15%-20% over the first 2-day period, no further release of DOX from either DOX@HAuNS or DOX@PEG-HAuNS was observed in water, phosphate-buffered saline (PBS, pH 7.0), or cell culture medium containing 10% fetal bovine serum over the second 2-day period (FIG. 10C). These results indicated that DOX was stably absorbed to HAuNS and PEG-HAuNS.

To investigate the mechanism of DOX binding to HAuNS, the amino group in DOX was blocked by an acetyl protecting group. The binding of DOX-acetamide to HAuNS was reduced to nearly zero (FIG. 10D), suggesting that the amino group of DOX was key for its high payload onto HAuNS. According to synthesis protocol, HAuNS were stabilized by negatively charged citrate with a zeta potential of −25 mA in PBS solution (pH 7.4). On the other hand, DOX was positively charged at pH 7.4. As a result, DOX was absorbed onto HAuNS via electrostatic interaction. The formation of complexes between drug molecules and nanocarriers is advantageous compared to covalent conjugation approach owing to easy fabrication and scale up, low cost, and predictable release profile. Gibson, J. D. et al., Paclitaxel-Functionalized Gold Nanoparticles, 129 (37), 11653-6, J Am Chem Soc (2007); Peer, D. et al., Nanocarriers as an Emerging Platform For Cancer Therapy, 2 (12), 751-60, Nat Nanotechnol (2007).

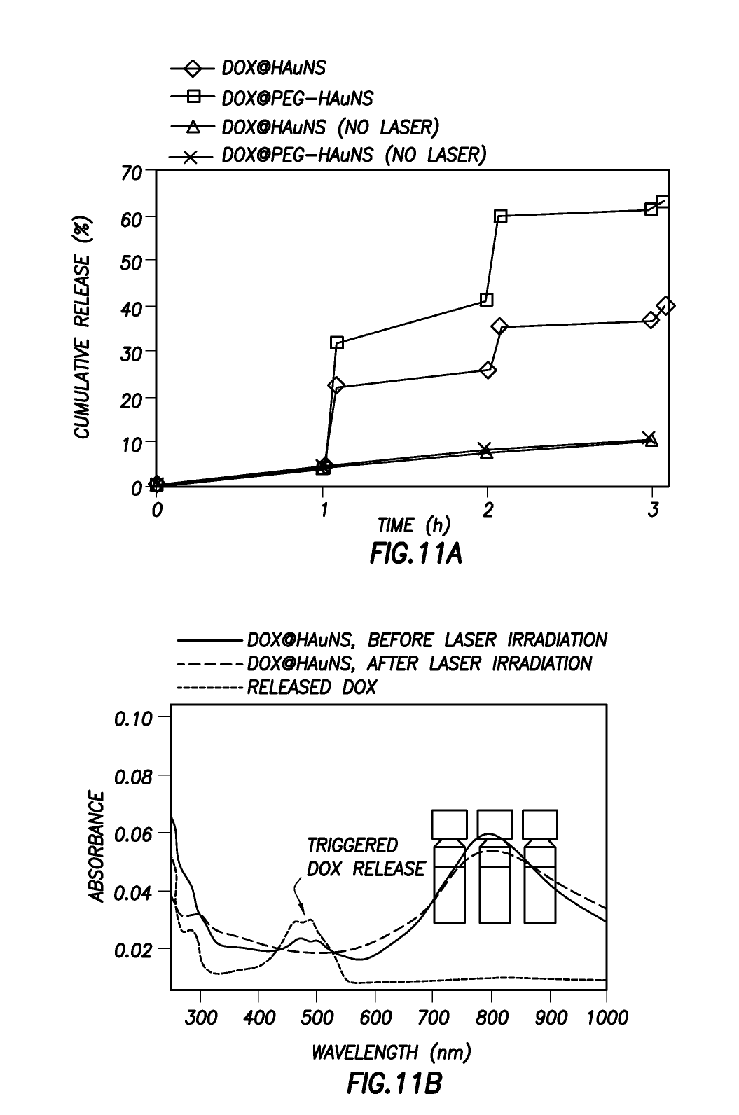

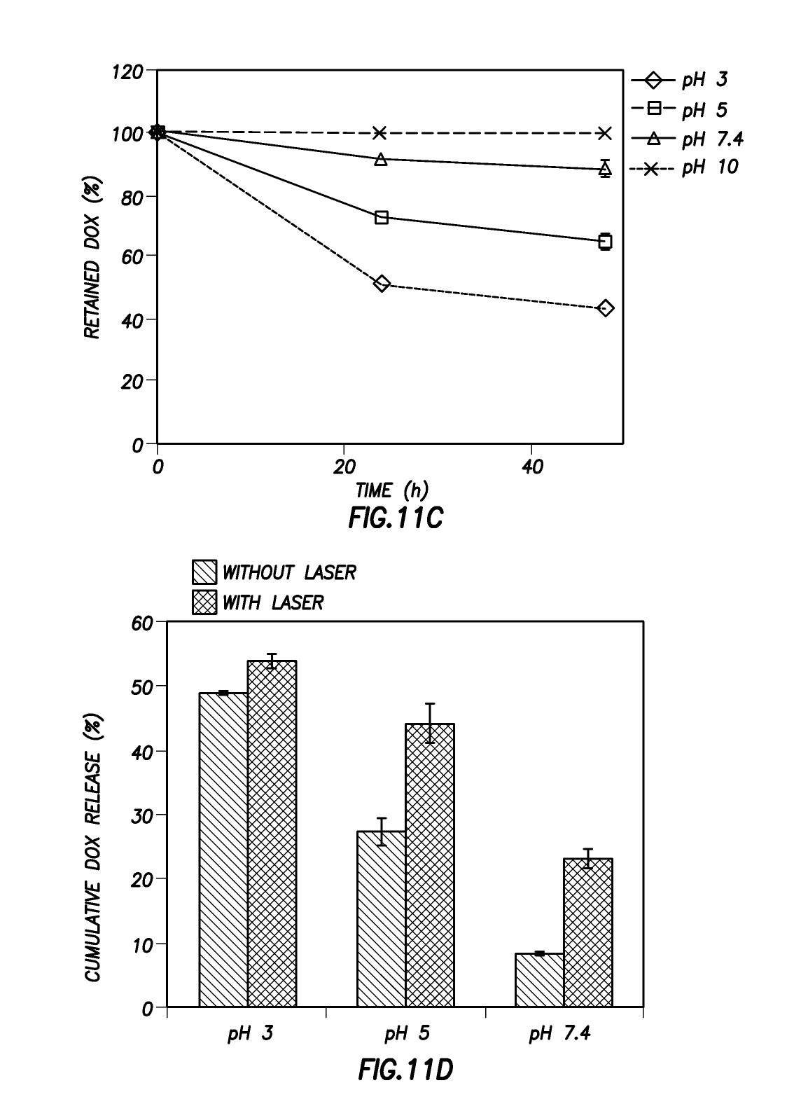

DOX release from DOX@HAuNS and DOX@PEG-HAuNS could be readily controlled by using NIR laser. After the first NIR laser irradiation (begun at 1 h) at 4.0 W/cm² output power for 5 min, the cumulative release, defined as the ratio of released DOX to total loaded DOX expressed as a percentage, increased from 4.1% to 22.2% for DOX@HAuNS and from 4.1% to 31.9% DOX for DOX@PEG-HAuNS (FIG. 11A). Release of DOX was significantly reduced or almost completely stopped when the NIR laser was switched off over the next 1 h of incubation. Similar results were observed when the laser treatment protocol was repeated beginning at 2 h. However, there was less DOX released during the second treatment cycle. By the third treatment cycle (beginning at 3 h), almost no DOX was released. These data suggested that DOX release from both DOX@HAuNS and DOX@PEG-HAuNS could be triggered by external NIR laser.

After NIR laser irradiation of DOX@HAuNS (˜1.0×10¹² particles/mL), the peak absorption intensity at around 490 nm was significantly increased, indicating release of free DOX from the nanocomplexes. The color of DOX@HAuNS changed from brown to green due to detachment of DOX from DOX@HAuNS (FIG. 11B). After centrifugal removal of free DOX, the remaining HAuNS solution displayed an absorption spectrum similar to that of DOX@HAuNS acquired prior to NIR laser irradiation but at a lower intensity, and with loss of the characteristic peak absorption of DOX at 490 nm (FIG. 11B). The reduced peak intensity at 800 nm after NIR laser irradiation may be attributed to fragmentation of a small fraction of HAuNS particles (data not shown).

Because DOX was attached to HAuNS via electrostatic interaction, it was anticipated that the release of DOX from DOX@HAuNS and DOX@PEG-HAuNS would be pH-dependent. Indeed, while there was no DOX released from DOX@PEG-HAuNS in PBS at pH 10 and only 11% DOX released in PBS at pH 7.4 at room temperature after 2 days of incubation, the DOX release reached 35% at pH 5.0 and 57% at pH 3.0 (FIG. 11C). The observed pH dependency is attributed to the increased hydrophilicity and higher solubility of DOX at lower pH caused by increased protonation of —NH₂ groups on DOX, which reduce the interaction between DOX and HAuNS. When the pH was decreased, the COO⁻ groups on the surface of HAuNS also became protonated. As a result, the electrostatic interaction between DOX and HAuNS was reduced. The pH-dependent drug release from HAuNS or PEG-HAuNS could be exploited for drug delivery applications: the microenvironments of extracellular tissues of tumors and intracellular lysosomes and endosomes are acidic, and this acidity could facilitate active drug release from HAuNS-based delivery vehicles.

NIR laser irradiation increased the amount of DOX released from DOX@HAuNS or DOX@PEG-HAuNS incubated in PBS at different pH levels; the lower the pH of the medium, the less DOX released. For example, after 5 min of continuous NIR laser irradiation at 4.0 W/cm², 14.6%, 16.7%, and 5.1% more DOX was released from DOX@HAuNS when the nanoparticles were incubated in PBS at pH 7.4, pH 5.0, and pH 3.0, respectively (FIG. 11D). The ability to achieve higher DOX release with NIR light at pH ˜5.0 is favorable because the intracellular lysosome environment of tumor cells has a pH of approximately 5.0. Ji, X. et al., Bifunctional Gold Nanoshells with a Superparamagnetic Iron Oxide-Silica Core Suitable for Both MR Imaging And Photothermal Therapy, 111 (17), 6245-6251, J Phys Chem C (2007).

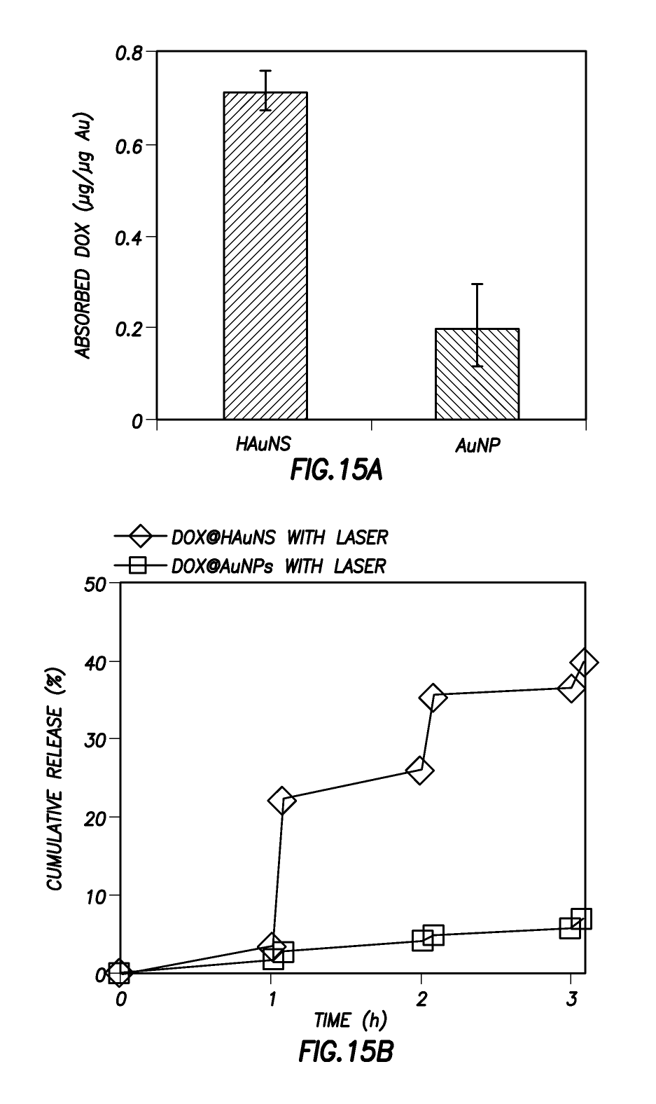

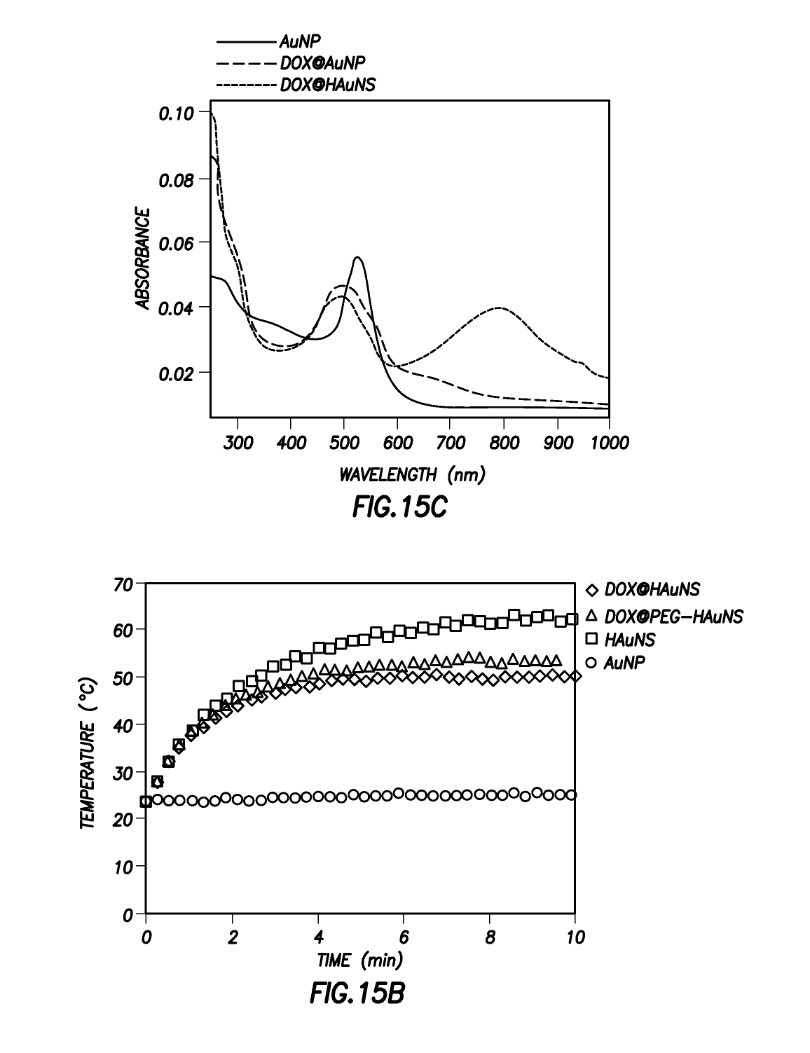

To explain the observed high loading capacity of DOX to HAuNS and further evaluate the advantages of HAuNS as drug carriers, we compared the DOX loading efficiency and drug release behavior between HAuNS and AuNPs having similar size (˜40 nm) and surface charge (zeta potential ˜−25 mA). HAuNS or AuNPs with the same equivalent Au concentration were incubated with DOX (final concentration of 1.0 mM). On the basis of 1.0 μg Au, the DOX payload increased from ˜0.2 μg in AuNPs to 0.7 μg in HAuNS, a 3.5-fold increase (FIG. 15A). This increase can be explained by the greater surface area of HAuNS compared to AuNPs. Assuming that the shell thickness of HAuNS is 4.0 nm and the diameter of both HAuNS and AuNP is 40 nm, it is estimated that each solid AuNP is equivalent to two hollow HAuNS on the basis of weight. This means that HAuNS should have twice the DOX payload of AuNPs if all DOX molecules are coated on the outer surface of HAuNS. The fact that 3.5-fold higher DOX was found bound to HAuNS suggests that the inner surface of HAuNS was also coated with DOX. In fact, it is estimated that HAuNS possess 3.2-fold greater surface area than AuNPs when the inner and outer surface areas are counted. TEM showed that the shell of HAuNS was porous (FIG. 9A), which makes it possible for DOX molecules to diffuse into the core and bind to the inner surface of HAuNS.

In addition to significant difference in DOX loading capacity between HAuNS and AuNP, DOX@HAuNS also displayed distinct characteristic of NIR light-triggered DOX release. In contrast, no DOX release was observed when DOX-coated AuNP was irradiated with NIR light (FIG. 15B). This is because unlike DOX@HAuNS, there was no plasmon absorption in the NIR region for DOX@AuNP (FIG. 15C). When aqueous solutions of HAuNS, DOX@HAuNS, and DOX@PEG-HAuNS having the same nanoparticle concentration of 0.7×10¹¹ particles/mL were exposed to NIR light (5.0 W/cm² for 10 min), the temperature was increased 39° C., 27° C., and 30° C., respectively. In comparison, no significant temperature change was observed when PBS or solution of AuNP was exposed to the NIR laser (FIG. 15D). Thus, the temperature elevation mediated by HAuNS in the presence of NIR light may be responsible for NIR laser-triggered release of DOX from DOX@HAuNS and DOX@PEG-HAuNS.

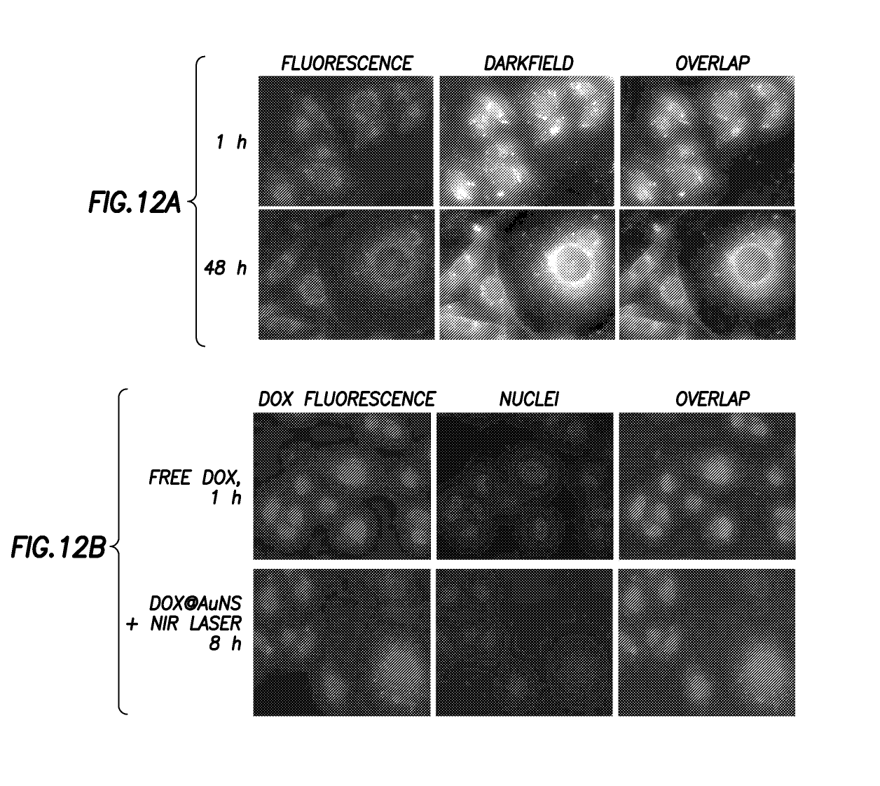

Both DOX@HAuNS and DOX@PEG-HAuNS were internalized into MDA-MB-231 cells and were retained in the endolysosomal compartments. After 1 h incubation, DOX@HAuNS showed strong red fluorescence signal from DOX despite quenching effect with DOX bound to HAuNS. The fluorescence signal was limited to spots scattered throughout the cytoplasm. The white bright dots obtained from dark-field imaging indicated the presence of HAuNS, which to a large extent colocalized with DOX (FIG. 12A). These results suggested that HAuNS together with DOX were phagocytosed by the cancer cells and distributed to the endolysosomal vehicles. After 48 h, DOX and HAuNS remained trapped in the endolysosomal vehicles (FIG. 12A). This observation contrasts with our earlier findings that DOX could be released from DOX@HAuNS at ˜pH 5. It may be that in the microenvironment of endolysosomes, the detached DOX re-attached to HAuNS before the drug molecules had a chance to diffuse out of the vehicles. In contrast to DOX@HAuNS, free DOX was taken up by the tumor cells and distributed to cell nuclei 1 h after incubation (FIG. 12B). NIR laser irradiation (1.0 W/cm² for 3 min per treatment, 4 treatments over a 2-h period) caused release of DOX from DOX@HAuNS, and DOX was distributed to cell nuclei (FIG. 12B). Thus, it is possible to control intracellular DOX release from DOX@HAuNS and DOX@PEG-HAuNS by NIR laser irradiation.

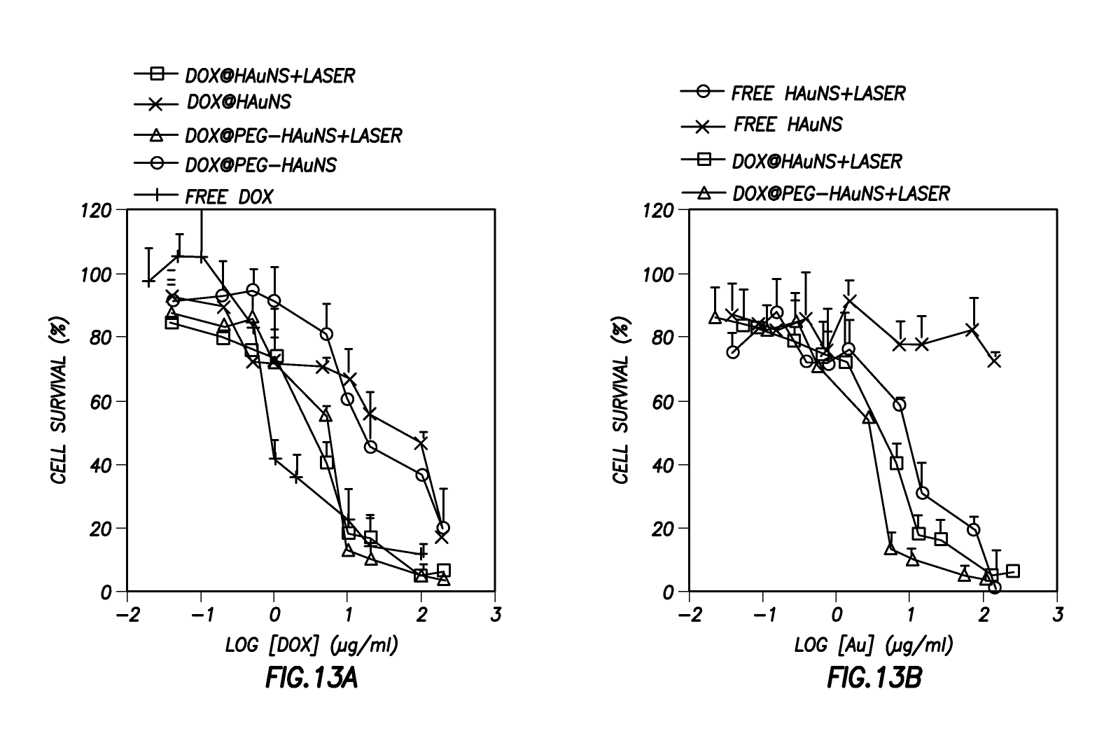

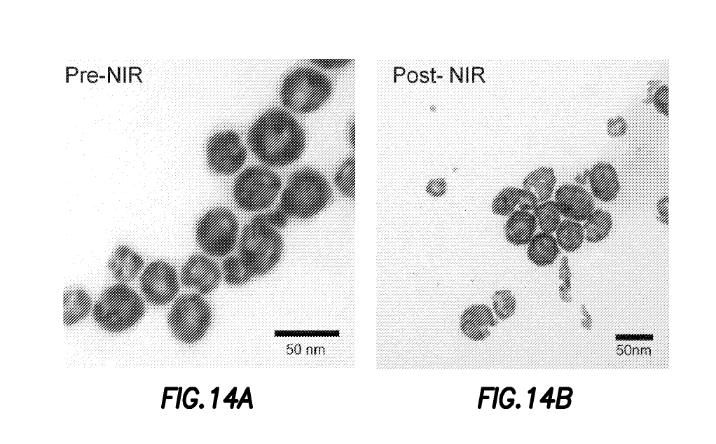

Both DOX@HAuNS and DOX@PEG-HAuNS were cytotoxic against MDA-MB-231 cells in a dose-dependent manner. About 33.5% and 39.5% of cells were killed by DOX@HAuNS and DOX@PEG-HAuNS, respectively at an equivalent DOX concentration of 10 μg/mL (FIG. 13A). However, free DOX exhibited higher toxicity, with 77.4% cell killed at the same drug concentration. The lower cell killing potency with DOX@HAuNS and DOX@PEG-HAuNS could be attributed to relatively stable complexes formed between DOX and HAuNS and delayed DOX release inside cells. After NIR laser irradiation (2 W/cm² for 3 min per treatment, 4 treatments over a 2-h period), both DOX@HAuNS and DOX@PEG-HAuNS showed significant enhanced cell-killing effect toward cancer cells, with about 83.5% and 86.4% cell killed, respectively, at an equivalent DOX concentration of 10 μg/mL (FIG. 13A). HAuNS was not cytotoxic with Au concentrations ranging from 0.04 μg/mL to 160 μg/mL (FIG. 13B). This is consistent with literature findings that in general, Au-based nanoparticles are well tolerated. De, M. et al., Applications of Nanoparticles in Biology, 20 (22), 4225-4241, Adv Mater (2008); Bhattacharya, R. et al., Biological properties of “Naked” Metal Nanoparticles, 60 (11), 1289-306, Adv Drug Deliv Rev (2008). Plain unloaded HAuNS exhibited significant cell-killing effect (>69% cells killed), when cells incubated with HAuNS were treated with NIR laser at Au concentrations greater than 16 μg Au/mL (FIG. 13B). These results indicate that HAuNS produced significant photothermal ablation at concentrations greater than 16 μg Au/mL. At an Au concentration of 5.9 μg/mL (10 μg/mL equivalent DOX), 86.4% of cells were killed with combined DOX@PEG-HAuNS and NIR laser treatments. In comparison, treatments with combined HAuNS and NIR laser killed only 40.6% of the cancer cells (FIG. 13B). The enhanced cytotoxicity of DOX@HAuNS and DOX@PEG-HAuNS at lower concentrations primarily resulted from DOX released upon NIR laser irradiation. Thus, at higher concentrations of DOX@HAuNS and DOX@PEG-HAuNS, both photothermal ablation and cytotoxic activity of DOX contributed to the killing of cancer cells.

We claim:

1. A drug delivery system comprising a microsphere that contains a plurality of hollow gold nanospheres and drug product, wherein upon NIR irradiation, the drug product is released from the microsphere.

2. The drug delivery system of claim 1, wherein the drug product is positively or negatively charged and forms a complex directly with the hollow gold nanosphere.

3. The drug delivery system of claim 1, wherein the microsphere is made of polymeric material.

4. The drug delivery system of claim 1, wherein the drug product is hydrophilic or hydrophobic and is incorporated within the microsphere with the plurality of hollow gold nanospheres in a polymeric matrix.

5. A method of making a near-infrared mediated drug delivery system comprising the step of forming a polymeric matrix of hydrophobic drug product and hollow gold nanospheres within a microsphere, wherein the hollow gold nanospheres are located within an inner water phase and the drug product is dispersed uniformly within the microsphere.

6. A method of making a near-infrared mediated drug delivery system comprising the step of forming a polymeric matrix of hydrophilic drug product and hollow gold nanospheres within a microsphere, wherein the hollow gold nanospheres and hydrophilic drug product are located within an inner water phase.

7. A method of making a near-infrared mediated drug delivery system comprising the step of forming a polymeric matrix of hydrophilic and hydrophobic drug products and hollow gold nanospheres within a microsphere, wherein the hollow gold nanospheres and hydrophilic drug product are located within an inner water phase, and the hydrophobic drug product is dispersed uniformly within the microsphere.

8. A polymeric microsphere useful in drug entrapment for NIR light mediated release, wherein the microsphere comprises a plurality of hollow gold spheres and one or more drug products, and the hollow gold spheres and drug products are loaded in the polymeric matrix.

9. A HAuNS-drug complex useful for NIR light mediated release, wherein drug product is complexed directly to HAuNS through electrostatic interaction.
